(19) United States
(12) Patent Application Publication
(10) Pub. No.: US 2004/0210037 A1
(43) Pub. Date: Oct. 21, 2004

(54) Targeted MHC class I alpha3 vaccine delivery systems

(75) Inventors: Maurice Zauderer, Pittsford, NY (US); Mark J. Paris, West Henrietta, NY (US); Ernest S. Smith, Ontario, NY (US)

Correspondence Address: STERNE, KESSLER, GOLDSTEIN & FOX PLLC, 1100 NEW YORK AVENUE, N.W., WASHINGTON, DC, 20005, US

(73) Assignee: Vaccinex, Inc., Rochester, NY

(21) Appl. No.: 10/809,790

(22) Filed: Mar. 26, 2004

Related U.S. Application Data
(60) Provisional application No. 60/457,896, filed on Mar. 28, 2003.

(51) Int. Cl. C07K 16/46

(52) U.S. Cl. 530/350; 530/391.1

(57) ABSTRACT

The present invention is directed to a novel targeted vaccine delivery system, comprising one or more isolated MHC class I α3 domains linked to an antibody which is specific for a cell surface marker and associated with β2-microglobulin and antigenic peptides, or costimulatory molecules, or cytokines. The complexes of the invention are useful for treating and/or preventing cancer, infectious diseases, autoimmune diseases, and/or allergies.

### BACKGROUND OF THE INVENTION

1. Field of the Invention

The present invention relates to immunology. More specifically, the present invention relates to vaccines and methods for modifying immune responses.

2. Related Art

T lymphocytes are both key effector cells and key regulatory cells of the immune system. The ability to stimulate or inhibit specific T cell responses is a major goal for the immunotherapy of cancer, infectious diseases, and autoimmune diseases. T cell specificity is mediated by a T cell receptor (TCR) on the surface of the T cells. Each TCR is specific for a complex of a unique peptide epitope of a protein antigen associated with a major histocompatibility complex (MHC) molecule on the surface of a cell. There are two classes of MHC proteins which bind to TCRs in conjunction with peptide antigens: MHC class I proteins, which are found on the membranes of all nucleated cells; and MHC class II proteins, which are found only on certain cells of the immune system. The two major classes of T cells, CD8+ and CD4+, are selected to be specific for peptide epitopes that associate, respectively, with MHC class I and class II molecules on the antigen presenting cell. Polymorphism within each class of MHC molecule determines which peptide fragments bind with functional affinity to the MHC molecules expressed by a particular individual.

The MHC class I molecule is comprised of a polymorphic a heavy chain non-covalently associated with non-polymorphic β2-microglobulin. The a heavy chain has three extracellular domains (designated α1, α2, and α3) in addition to transmembrane and cytoplasmic domains. It has been determined by X-ray crystallography that the highly polymorphic α1and α2 domains form a peptide binding groove. The relatively non-polymorphic α3 domain connects the α1:α2 peptide binding structure to the transmembrane region and is structurally homologous to an immunoglobulin constant region domain. β2-microglobulin interacts both with the underlying surface of the α1:α2 structure and with the nonpolymorphic α3 domain. It has been demonstrated that the isolated MHC class I α3 domain can be produced as an independent functional unit dissociated from the α1, α2, transmembrane and cytoplasmic domains of the MHC class I heavy chain. Importantly, the α3 domain folds properly in the absence of flanking sequences within the α heavy chain and retains the ability to associate non-covalently with β2-microglobulin. The equilibrium dissociation constant (Kd) for β2-microglobulin binding to the isolated MHC class I α3 domain is in the micromolar range whereas binding to intact MHC class I α chain has an equilibrium constant in the nanomolar range. See Whitman, M. C. et al., Mol. Immunol. 37:141-9 (2000); Parker, K. C. et al., Biochem. 24:5543-50 (1985); and Hochman, J. H. et al., J. Imnmunol. 140:2322-29 (1988). It has been suggested that affinity for the isolated α3 domain accounts for the initial binding of β2-microglobulin to an independently folded α3 domain in the intact αchain and that the enhanced affinity due to interaction with the α1:α2 structure promotes proper folding of the αchain and helps stabilize peptide binding. See Hebert, A. M. et al., Biochem. 40:5233-42 (2001).

Binding of peptide-MHC complexes to T cells is, in general, not sufficient to induce T cell proliferation and differentiation. Additional costimulatory signals delivered through interactions between other membrane molecules of the T cell and the antigen presenting cell are required for optimal T cell activation. Indeed, signaling through T cell antigen receptor alone in the absence of costimulation can result in tolerization rather than activation.

Methods are available to target a specific ligand or regulatory molecule to an antigen positive cell by genetically linking the specificity domain of an antibody specific for that antigen to a particular ligand or cytokine. Fusion proteins encoded in this fashion may retain both antigen specificity and ligand or cytokine function. Examples of such reagents have been described in which the ligand coding sequence is linked to either the carboxyl or amino terminus of an antibody chain which may itself be either whole or truncated (Morrison, S. L. et al., Clin. Chem. 34:1668-1675 (1988); Shin, S. U. and Morrison, S. L., Meth. in Enzymol. 178:459-476 (1989); Porto, J. D. et al., Proc. Nat'l. Acad. Sci. USA 90:6671-6675 (1993); Shin, S.-U. et al., J. Immunol. 158:4797-4804 (1997)). A particularly flexible construct has been described, in which an avidin molecule is linked to the carboxyl-terminus of the heavy chain of an antibody that can target the transferrin receptor and can, in principle, deliver any biotinylated ligand to the target cell (Penichet, M. L. et al., J. Immunol. 163:4421-4426 (1993)).

The key requirements for construction of a delivery system that can target specific cells and tissues to deliver a ligand or cytokine are to identify an appropriate target molecule, select an antibody with a specificity domain with high affinity for that target molecule, and to link an effective concentration of ligand or cytokine to that antibody specificity domain. For the specific purpose of vaccine delivery to stimulate CD8+ cytotoxic T cells or sensitize target cells to such T cells, the relevant ligand is a peptide in a form which is efficiently expressed as a complex with MHC class I molecules on the cell surface. An especially useful delivery vehicle would include a specific peptide which could be targeted to professional antigen presenting cells, such as dendritic cells, or other cells, such as tumor cells, epithelial cells, fibroblasts, lymphoid cells including activated or transfomied T cells, or infected cells, and deliver an effective concentration of peptide complex to modulate (i.e., stimulate or inhibit) a specific T cell response.

In view of the diversity of antigens expressed in cancer and in infectious or autoimmune disease, and the natural polymorphism of human MHC, effective use of such fusion proteins for immunotherapy would be greatly facilitated by the ability to flexibly couple different multimeric, functional fragments of MHC complexes to one or more cell targeting specificities.

### SUMMARY OF THE INVENTION

The present invention provides compounds useful for modulating, i.e., either inhibiting or stimulating, an immune response. The compound of the invention comprises one or more isolated MHC class I α3 domains linked to an antibody or fragment thereof specific for a cell surface marker. Preferably, the isolated MHC class I α3 domains are linked to the carboxyl terminus of the antibody or fragment thereof.

In one embodiment, the compound comprises one or more MHC class I α3 domains linked to an antibody or fragment thereof specific for a cell surface marker. The MHC-peptide complexes linked to antibody or antibody fragments comprise an MHC class I α3 domain, a β₂-microglobulin molecule or fragment thereof, and an antigenic peptide. In certain embodiments, the antigenic peptide is covalently linked to the β₂-microglobulin molecule or fragment thereof.

In another embodiment, the MHC class I α3 complexes comprise an MHC class I α3 domain, a β₂-microglobulin molecule or fragment thereof, and a costimulatory molecule. In certain embodiments, the costimulatory molecule is linked to the β₂-microglobulin molecule or fragment thereof.

In certain embodiments, the MHC class I α3 complexes are linked to the antibody or fragment thereof through a multivalent compound.

In certain embodiments, the antibody is specific for a cell surface marker of a professional antigen presenting cell, more particularly a dendritic cell. In other embodiments, the antibody is specific for a cell surface marker of a tumor cell, an epithelial cell or a fibroblast. In other embodiments, the antibody is specific for a cell surface marker of a T cell. In other embodiments, the antibody is specific for a cell surface marker of an infected cell.

In certain embodiments, the antigenic peptide is derived from a cancer cell. In other embodiments, the antigenic peptide is derived from an infectious agent or an infected cell. In still other embodiments, the antigenic peptide is derived from an allergen or the target tissue of an autoimmune disease. In other embodiments, the antigenic peptide is synthetic.

Also provided are methods of modulating, i.e., either stimulating or inhibiting, an immune response, comprising administering to an animal an effective amount of a compound or composition of the invention.

### BRIEF DESCRIPTION OF THE FIGURES

FIG. 1 shows the nucleotide sequence of HLA-A*0201 (SEQ ID NO:1).

FIG. 2 shows the nucleotide sequence of the assembled Ig Gamma Heavy Chain (SEQ ID NO:2). Double underline: NotI restriction site; single underline: signal sequence; bold: BssHII restriction site; dashed underline: BstEII restriction site.

FIG. 3 shows the nucleotide and amino acid sequence of the Ig-α3 assembled chimera (SEQ ID NOs:3 and 4). Double underline: NotI restriction site; single underline: Signal sequence; bold: BssHII restriction site; dashed underline: BstEII restriction site; and wavy underline: BamHI restriction site. The variable gene sequence is introduced at the histidine in bold and the rest of the bolded amino acids are removed upon insertion of the variable gene sequence.

FIG. 4 shows the nucleotide and amino acid sequence of the CMV peptide-β₂-microglobulin chimera (SEQ ID NOs:5 and 6).

### DETAILED DESCRIPTION OF THE INVENTION

The present invention provides compounds which are useful for modulating, i.e., either inhibiting or stimulating, an immune response. The compounds comprise one or more MHC class I α3 complexes linked to an antibody or fragment thereof specific for a cell surface marker. The compounds are useful for stimulating desirable immune responses, for example, immune responses against infectious agents or cancer; or for inhibiting undesirable immune responses, such as allergic responses, allograft rejections, and autoimmune diseases. The present invention targets a MHC class I α3 complex to professional antigen presenting cells, such as dendritic cells, B cells, or macrophages; or other class I MHC positive cells such as tumor cells; epithelial cells; fibroblasts; T cells; or other cells, by linking one or more MHC class I α3 complexes to an antibody or fragment thereof specific for a surface antigen of the targeted cell type. Depending on the targeted cell type, this will lead to either very efficient stimulation or inhibition of antigen specific T cell activity.

MHC class I molecules consist of an α (heavy) chain, coded for by MHC genes, associated with β₂-microglobulin, coded for by non-MHC genes. As described above, the α₃ segment of the heavy chain can fold as an isolated domain and bind to β₂-microglobulin protein; the α₁ and α₂ regions of the heavy chain form the base and walls of the antigen-binding pocket (Science 238:613-614(1987); Bjorkman, P. J. et al., Nature 329:506-518 (1987)). An α chain may come from genes in the HLA-A, B or C subgroups. Class I molecules bind peptides of about 8-10 amino acids in length. All humans have between three and six different classical class I molecules, which can each bind many different types of peptides.

A fragment of the complete human HLA-A*0201 MHC class I αchain sequence has been described that folds independently into an MHC class I α3 domain and binds β2-microglobulin, Fayen, J. et al. Mol. Immunol. 32:267 (1995). In what follows any homologous MHC class I αchain fragment that is either (i) identical to this previously described MHC class I α3 domain sequence or (ii) has substitutions of less than 1, 2, 3, 4, 5, 6, 7, 8, 9, 10, 11, 12, 13, 14, 15, 16, 17, 18, 19 or 20 amino acids which result in no more than a factor of 10 reduction in affinity for β2-microglobulin or (iii) extends further into the transmembrane and/or the α2 domain of the native a chain sequence and to which β2-microglobulin binds with an affinity that remains less than one tenth the binding affinity of β2-microglobulin for the intact MHC class I α chain or (iv) is shorter by any amount which is still compatible with no more than a factor of 10 reduction in affinity for β2-microglobulin will be referred to as an MHC class I α3 domain.

The compounds of the invention comprise one or more MHC class I α3 complexes linked to an antibody or fragment thereof, wherein the antibody is specific for a cell surface marker. The MHC class I α3 complexes of the invention comprise the isolated MHC class I α3 domain, but not the complete α1, α2, transmembrane or cytoplasmic domains of the MHC class I α chain.

The complexes also comprise β₂-microglobulin. The β₂-microglobulin of the complex associates with the isolated MHC class I α3 domain. However, because of additional interactions with the α1 and β2 domains, β₂-microglobulin has higher affinity for the full length MHC class I αchain than for the isolated α3 domain. Thus, when the compounds of the invention are delivered to cells bearing MHC class I, it is thermodynamically favored for the β₂-microglobulin of the inventive compounds to “pop off” and preferentially associate with the complete αchain of native full length MHC class I present on the surface of the cells. To further drive favorable energetics for the displacement of β₂-microglobulin already associated with the class I molecule, the β₂-microglobulin present in the compounds of the invention may be altered in such a way as to have higher affinity for the complete MHC α chain than native β₂-microglobulin. See Ribaudo, R. K. et al., WO 99/64597, published 16 Dec. 1999, the disclosure of which is incorporated by reference herein. This altered β₂-microglobulin would then have greater likelihood of displacing the native β₂-microglobulin. In addition, for those embodiments in which β₂-microglobulin is linked to a peptide which can associate with the peptide binding groove formed by the α1 and α2 domains of the intact α chain, the cumulative avidity of β₂-microglobulin linked to peptide for that α chain will be significantly greater than the affinity for either β₂-microglobulin alone or peptide alone. This will further favor displacement by the linked peptide-β₂-microglobulin of both native β₂-microglobulin and natural peptides bound to the intact class I MHC molecule.

In comparison with compounds which comprise the entire MHC molecule, the compounds of the present invention are immunologically relevant to a wider variety of individuals all of whom may associate the same peptide with different MHC class I molecules, particularly the related MHC class I molecules of a single supertype. See Sidney, J. et al., Immunol. Today 17:261 (1996) and Sidney, J. et al., Human Immunol. 62:1200-16. Since the β₂-microglobulin and MHC class I α3 domain are non-polymorphic, the compounds of the invention should not induce an immune response against themselves in most subjects.

The MHC molecules (both the α3 and β₂-microglobulin portions) useful in the present invention may be autologous to any mammalian or avian species, for example, primates (esp. humans), rodents, rabbits, equines, bovines, canines, felines, etc. The term “MHC” encompasses similar molecules in different species. In mice, the MHC is termed H-2, in humans it is termed HLA for “Human Leucocyte Antigen”. When used herein, “MHC” is universally applied to all species. β₂-microglobulin is typically not inflammatory in vivo. However, it is preferable to employ β₂-microglobulin derived from the same species as is to be vaccinated so as to reduce the risk of a xenogeneic immune response.

Conventional identifications of particular MHC variants are used herein. For example, HLA-B 17 refers to a human leukocyte antigen from the B gene group (hence a class I type MHC) gene position (known as a gene locus) number 17.

In one embodiment, the MHC class I α3 complex comprises an MHC class I α3 domain, a β₂-microglobulin molecule or fragment thereof, and an antigenic peptide. The antigenic peptide may be linked to the β₂-microglobulin molecule or fragment thereof. Preferably, the antigenic peptide is covalently bound to the amino terminus of the β₂-microglobulin molecule or fragment thereof.

In another embodiment, the MHC class I α3 complex comprises an MHC class I α3 domain, a β₂-microglobulin molecule or fragment thereof, and an protein with immunological activity. Preferably, the protein with immunological activity is a costimulatory molecule, such as B7.1 or B7.2. “B7” is used herein to generically refer to either B7.1 or B7.2. Additionally, the protein with immunological activity may be a lymphokine or cytokine that modulates immune cell activation such as interleukins IL-1, IL-2, IL-3, IL-4, IL-5, IL-6, IL-12, IL-15, IL-18, or granulocyte-macrophage colony stimulating factor (GM-CSF), or transforming growth factor (TGF, e.g., TGFα and TGFβ), or lymphocyte function-associated protein, such as LFA-1 or LFA-3, or an intercellular adhesion molecule, such as ICAM-1 or ICAM-2.

In certain embodiments, the antibody or antibody fragment linked to MHC class I α3 complexes of the invention comprise more than one MHC class I α3 domain. These MUC class I α3 domains may be linked to each other and to the same or different immunoglobulin chains. This provides multiple sites for β₂-microglobulin molecules or fragments thereof to associate. Any number of MHC class I α3 domains may be linked together.

Preferably, 20, 19, 18, 17, 16, 15, 14, 13, 12, 11, 10, 9, 8, 7, 6, 5, 4, 3 or 2 MHC class I α3 domains are linked together. The MHC class I α3 domains may be directly fused, or may have a linker or spacer sequence between each molecule. The complex may also contain more than one β₂-microglobulin molecule or fragment thereof. Preferably, the complex contains 20, 19, 18, 17, 16, 15, 14, 13, 12, 11, 10, 9, 8, 7, 6, 5, 4, 3 or 2 β₂-microglobulin molecules or fragments thereof. Each β₂-microglobulin molecule or fragment thereof may be linked to the same antigenic peptide, costimulatory molecule, or other immunologically active protein. Alternatively, the complex may contain multiple β₂-microglobulin molecules or fragments thereof linked to different antigenic peptides and/or costimulatory molecules or immunologically active proteins.

In addition to β₂-microglobulin, a second non-polymorphic ligand that binds to different site on the α3 domain of the MHC class I heavy chain is the CD8 co-receptor molecule expressed on some T cells. It has been shown that mutations at amino acid positions 223 or 227 of the α3 domain of the MHC class I heavy chain interfere with binding to CD8. See Salter, R. D. et al., Nature 345:41-46 (1990). Isolated MHC class I α3 domains have also been shown to bind CD8. Therefore, an incidental and potentially undesirable effect of targeting multimeric MHC class I α3 domains to a target cell is that isolated MHC class I α3 domains may compete with intact MHC class I α chains of the target cell for interaction with the CD8 coreceptor of T cells. As suggested by Tykocinski, M. L. in WO 94/25610, published 10 Nov. 1994 (the disclosure of which is incorporated by reference herein) this could have the effect of inhibiting rather than stimulating T cell activity. To avoid this effect, the MHC class I α3 domains incorporated into compounds of the present invention may be mutated at the residues corresponding to amino acid residues 223 or 227 of the full length MHC class I αchain. See Salter, R. D. et al., Nature 345:41-46 (1990).

There are a number of mechanisms independent of MHC class I α3 domains whereby compounds of the invention could have an inhibitory effect on T cell activation. It has been reported that a lysine to glutamic acid mutation at position 58 of β2-microglobulin significantly reduces binding to the CD8 co-receptor and is a potent antagonist of CD8 dependent T cell activation. See Glick M et al. J. Biol. Chem. 277:20840-46 (2002). Compounds of the present invention afford a means of specifically delivering this inhibitory signal to target cells of an autoimmune or graft rejection response by linking MHC class I α3 complexes that comprise mutant K58E β2-microglobulin to an antibody specific for a target cell antigen. Other mechanisms of inhibition include delivery of anti-inflammatory cytokines or of altered peptide ligands with antagonist activity for specific T cell activation.

Preferably, the MHC class I α3 complexes are linked to the antibody through the MHC class I α3 domain. The MHC class I α3 domain may be linked to either the light chain or the heavy chain of the antibody, either directly or indirectly.

The MHC class I α3 complexes may be linked to either the carboxyl or amino terminus of the antibody, or they may be linked to the antibody at a site other than the carboxyl or amino termini. Preferably, the MHC class I α3 complexes are linked to the carboxyl terminus of the antibody.

Preferably, there are two MHC class I α3 complexes per antibody.

The attachment of the MHC chains to the antibody chains may be direct, i.e., without any intermediate sequence, or through a linker amino acid sequence, a linker molecule, or a chemical bond.

In certain embodiments, the MHC class I α3 domain may be linked in a fusion protein with the antibody. Fusion antibodies can be made using conventional recombinant nucleic acid techniques. The fusion may be direct or may contain spacers. The fusion proteins are comprised of an MHC class I α3 domain attached to the carboxyl terminus of an antibody or fragment thereof, wherein the antibody or fragment thereof is specific for a cell surface marker. Methods of making MHC-antibody fusion proteins are described in, for example, Dal Porto et al., Proc. Natl. Acad. Sci. USA 90:6671-6675 (1993) and Hamad et al., J. Exp Med. 188:1633-1640 (1998).

In another embodiment, the MHC class I α3 complexes are linked to the antibody through a multivalent compound. The MHC class I α3 complexes may be linked to the multivalent compound through any site. For example, the MHC class I α3 complexes may be linked through the amino or carboxyl terminus of the MHC class I α3 domain. Preferably, the complexes are linked to the multivalent compound through the carboxyl terminus of the α3 domain. These compounds comprise 2 or more MHC class I α3 complexes. The compounds may comprise 2, 3, 4, 5, 6, 7, 8, 9 or 10 MHC class I α3 complexes.

Likewise, the antibody may be linked to the multivalent compound through any site. The antibody may be linked to the multivalent compound through the light chain, the heavy chain, both light chains, both heavy chains, one light chain and one heavy chain, or both light and both heavy chains. Preferably, the antibody is linked to the multivalent compound at the carboxyl terminus.

Examples of multivalent compounds are chicken avidin or streptavidin (Shin, S. U. et al., J. Immunology 158: 4797-4804 (1997)) to which biotinylated MHC complexes are bound (Altman, J. et al, Science 274:94-96 (1996); Boniface, J. J. et al., Immunity 9:459-66 (1998)); or a leucine zipper system. Cochran, J. R. et al., Immunity 12:241-50 (2000) describe the use of chemically synthesized peptide-based cross-linking reagents in which two or more thiol-reactive maleimide groups are linked to lysine side chains in a flexible peptide of 8 to 19 residues containing glycine, serine, and glutamic acid in addition to the modified lysine residues. An isolated MHC class I α3 domain is modified to introduce a cysteine residue at the carboxyl terminus. Cysteine modified MHC class I α3 domains react with the maleimide groups on the various peptide backbones with either two, three, or four modified lysine residues for formation of MHC dimers, trimers, and tetramners. Pack, P. et al. J. Mol. Biol. 246:28-34 (1995) constructed tetravalent miniantibodies by fusing a modified GCN4-zipper that results in formation of highly stable trimeric and tetrameric structures to the carboxyl terminus of a single-chain Fv fragment via a flexible hinge region.

Yet another means of assembling polymeric MHC complexes on specific antibody is to exploit the observation that defined amino acid substitutions in the GCN4 leucine zipper dimerization domain results in formation of highly stable trimeric and tetrameric structures of the synthetic peptide (Harbury, P. B. et al., Science 262:1401-7 (1993)). Pack, P. et al. J. Mol. Biol. 246:28-34 (1995) constructed tetravalent miniantibodies by fusing the modified GCN4-zipper to the carboxyl terminus of a single-chain Fv fragment via a flexible hinge region. Several additional modifications of the fusion protein improved yield from bacterial synthesis. Addition of a carboxyl terminal tag would facilitate purification. Targeted tetravalent MHC complexes could be assembled from a mixture of single chain antibody and MHC class I α3 complexes each separately fused through a hinge region to the modified GCN4-zipper motif.

The compound of the invention may further comprise a cytokine or lymphokine. The cytokine or lymphokine may be linked to the multivalent compound, the antibody, or the MHC class I α3 complex. For example, the multivalent compound may be avidin or streptavidin, and the cytokine or lymphokine may be biotinylated. Alternatively, the cytokine or lymphokine may be directly fused to the antibody or MHC class I α3 complex.

Cytokines or lymphokines useful in the present invention include, but are not limited to, interleukins (e.g.., IL-2, IL-3, IL-4, IL-5, IL-6, IL-10, IL-12, IL-15, and IL-18), α interferons (e.g.., IFNα), β interferons (e.g.., IFNβ), γ interferons (e.g.., IFNγ), granulocyte-macrophage colony stimulating factor (GM-CSF), and transforming growth factor (TGF, e.g.., TGFα and TGFβ).

The compound of the invention may further comprise other therapeutic agents. The therapeutic agent or agents may be linked to the multivalent compound, the antibody, or the MHC class I α3 complex. Examples of therapeutic agents include, but are not limited to, antimetabolites, alkylating agents, anthracyclines, antibiotics, and anti-mitotic agents. Antimetabolites include methotrexate, 6-mercaptopurine, 6-thioguanine, cytarabine, 5-fluorouracil decarbazine. Alkylating agents include mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum(II) (DDP) cisplatin. Anthracyclines include daunorubicin (formerly daunomycin) and doxorubicin (also referred to herein as adriamycin). Additional examples include mitozantrone and bisantrene. Antibiotics include dactinomycin (formerly actinomycin), bleomycin, mithramycin, and anthramycin (AMC). Antimytotic agents include vincristine and vinblastine (which are commonly referred to as vinca alkaloids). Other cytotoxic agents include procarbazine, hydroxyurea, asparaginase, corticosteroids, mytotane (O,P′-(DDD)), interferons. Further examples of cytotoxic agents include, but are not limited to, ricin, doxorubicin, taxol, cytochalasin B, gramicidin D, ethidium bromide, etoposide, tenoposide, colchicin, dihydroxy anthracin dione, 1-dehydrotestosterone, and glucocorticoid.

Analogs and homologs of such therapeutic and cytotoxic agents are encompassed by the present invention. For example, the chemotherapuetic agent aminopterin has a correlative improved analog namely methotrexate. Further, the improved analog of doxorubicin is an Fe-chelate. Also, the improved analog for 1-methylnitrosourea is lomustine. Further, the improved analog of vinblastine is vincristine. Also, the improved analog of mechlorethamine is cyclophosphamide.

The MHC class I α3 domains may contain some amino acids from the α2 domain or the transmembrane domain. Preferably, not more than about 20, 19, 18, 17, 16, 15, 14, 13, 12, 11, 10, 9, 8, 7, 6, 5, 4, 3, 2 or 1, and preferably none of the amino acids of the α2 domain or the transmembrane domain will be included.

Alternatively, a portion of the MHC class I α3 domain may be deleted. The deletion may extend as much as about 20, 19, 18, 17, 16, 15, 14, 13, 12, 11, 10, 9, 8, 7, 6, 5, 4, 3, 2 or 1 amino acids into the α3 domain, preferably none of the amino acids of the α3 domain will be deleted.

Additionally, fragments of β₂-microglobulin are useful in the present invention. To be useful in the present invention, the fragment of β2-microglobulin would have to retain the ability to associate with the MHC class I α3 domain and, preferably, retain the ability to associate with other domains of the intact α chain. Preferably, not more than about 20, 19, 18, 17, 16, 15, 14, 13, 12, 11, 10, 9, 8, 7, 6, 5, 4, 3, 2 or 1, and preferably none of the amino acids of β₂-microglobulin will be deleted.

One may wish to introduce a small number of amino acids at the polypeptide termini of either the MHC class I α3 domain or the 2-microglobulin, usually not more than 20, 19, 18, 17, 16, 15, 14, 13, 12, 11, 10, 9, 8, 7, 6, 5, 4, 3, 2 or 1. The deletion or insertion of amino acids will usually be as a result of the needs of the construction, providing for convenient restriction sites, addition of processing signals, ease of manipulation, improvement in levels of expression, or the like. In addition, one may wish to substitute one or more amino acids with a different amino acid for similar reasons, usually not substituting more than about 10, 9, 8, 7, 6, 5, 4, 3, 2 or 1 amino acids in any one domain.

Additionally, β₂-microglobulin proteins which include amino acid substitutions are useful in the present invention. One particular amino acid substitution, serine to valine at position 55 of the mature β₂-microglobulin, has been described. See Ribaudo R K et al. PCT Application WO 99/64597, published 16 Dec. 1999, the disclosure of which is incorporated by reference herein. This serine to valine mutation increases the affinity of β2-microglobulin for full length MHC class I α chain.

The α3 chain and β₂-microglobulin may be separately produced and allowed to associate to form a stable heteroduplex complex (see Altman et al. (1993), or Garboczi et al. (1992)), or both of the subunits may be expressed in a single cell.

MHC molecules and fusion proteins useful in the compounds of the present invention may be isolated from a multiplicity of cells, e.g., transformed cell lines JY, BM92, WIN, MOC, and MG, and CHO using a variety of techniques known to those skilled in the art.

The amino acid sequence of a number of MHC proteins are known, and the genes have been cloned, therefore, the proteins can be made using recombinant methods. For example, the α3 domain of an MHC class I molecule, is synthesized and the amino termini coding sequence can be arbitrarily chosen to facilitate the ligation of the coding region for an antibody chain or fragment or a binding intermediate. The coding sequence for the α3 and β2 microglobulin chains or their fusion products are then inserted into expression vectors, expressed separately in an appropriate host, such as E. coli, yeast, insect cells, mammalian cells or other suitable cells, and the recombinant proteins obtained are recombined in the presence of the peptide antigen. Known, partial and putative MHC class I amino acid and nucleotide sequences, including the consensus sequence, are published (see, e.g., Zemmour and Parham, Immunogenetics 33:310-320 (1991)), and cell lines in which fusion proteins may be expressed are known and generally available as well, many from the American Type Culture Collection (“ATCC”).

Antigenic peptides useful within the present invention include any peptide which is capable of modulating an immune response in an animal when presented in conjunction with an MHC molecule. Peptides may be derived from foreign antigens or from autoantigens.

The antigenic peptide will be from about 6 to 12 amino acids in length, usually from about 8 to 10 amino acids, most preferably 9 amino acids. However, the antigenic peptide may be 6, 7, 8, 9, 10, 11 or 12 amino acids in length.

Methods for determining whether a particular peptide will bind to a particular MHC molecule are known in the art. See, for example, Parker et al., J. Immunol. 149:3580-3587 (1992); Southwood et al., J. Immunol. 160:3363-3373 (1998); Sturniolo et al., Nature Biotechnol. 17:5555-560 (1999).

The compounds of the invention may contain a homogenous or heterogeneous population of peptides and/or costimulatory molecules. That is, each β2 microglobulin in the compound may be linked to the same antigenic peptide or each β2 microglobulin may be linked to different peptides.

Likewise, each β2 microglobulin may be linked to the same costimulatory molecule or each β2 microglobulin may be linked to different costimulatory molecules. Alternatively, some of the β2 microglobulins may be linked to an antigenic peptide, while some of the β2 microglobulins may be linked to a costimulatory molecule.

Peptides according to the present invention may be obtained from naturally-occurring sources or may be synthesized using known methods. For example, peptides may be synthesized on an Applied Biosystems synthesizer, ABI 431A (Foster City, Calif.) and subsequently purified by HPLC. Alternatively, DNA sequences can be prepared which encode the particular peptide and may be cloned and expressed to provide the desired peptide. In this instance a methionine may be the first amino acid. In addition, peptides may be produced by recombinant methods as a fusion to a protein that is one of a specific binding pair, allowing purification of the fuision protein by means of affinity reagents, followed by proteolytic cleavage, usually at an engineered site to yield the desired peptide (see for example Driscoll et al., J. Mol. Bio. 232:342-350 (1993)). The peptides may also be isolated from natural sources and purified by known techniques, including, for example, chromatography on ion exchange materials, separation by size, immunoaffinity chromatography and electrophoresis.

Isolation or synthesis of “random” peptides may also be appropriate, particularly when one is attempting to ascertain which particular epitope is most likely to stimulate T cells. One may produce a mixture of “random” peptides via use of proteasomes or by subjecting a protein or polypeptide to a degradative process—e.g., digestion with chymotrypsin—or peptides may be synthesized.

If one is synthesizing peptides, e.g., random 8-, and 9-amino acid peptides, all varieties of amino acids are preferably incorporated during each cycle of the synthesis. It should be noted, however, that various parameters—e.g., solvent incompatibility of certain amino acids—may result in a mixture which contains peptides lacking certain amino acids. The process should thus be adjusted as needed—i.e., by altering solvents and reaction conditions—to produce the greatest variety of peptides.

A number of computer algorithms have been described for identification of peptides in a larger protein that may satisfy the requirements of peptide binding motifs for specific MHC class I molecules. Because of the extensive polymorphism of MHC molecules, different peptides will often bind to different MHC molecules.

In one embodiment, the antigenic peptide is derived from a cancerous cell, or promotes an imnmune response against a cancerous cell.

Table 1 lists peptides of the C35 breast cancer antigen predicted for binding to the HLA class I molecule HLA-A*0201 as well as a few limited examples of C35 peptides that express binding motifs specific for other selected class I MHC molecules. Other C35 peptides which bind to specific HLA molecules are predicted in U.S. Appl. Publ. No. 2002/0155447 A1, published Oct. 24, 2002, the disclosure of which is incorporated by reference herein.

TABLE 1
Predicted C35 MHC Class I epitopes*

| | | |
|---|---|---|
| HLA | | |
| restriction | Inclusive | |
| element | amino acids | Sequence |

| | | | |
|---|---|---|---|
| A*0201 | 9-17 | SVAPPPEEV | (SEQ ID NO:7) |
| A*0201 | 10-17 | VAPPPEEV | (SEQ ID NO:8) |
| A*0201 | 16-23 | EVEPGSGV | (SEQ ID NO:9) |
| A*0201 | 16-25 | EVEPGSGVRI | (SEQ ID NO:10) |
| A*0201 | 36-43 | EATYLELA | (SEQ ID NO:11) |
| A*0201 | 37-45 | ATYLELASA | (SEQ ID NO:12) |
| A*0201 | 37-46 | ATYLELASAV | (SEQ ID NO:13) |
| A*0201 | 39-46 | YLELASAV | (SEQ ID NO:14) |
| A*0201 | 44-53 | SAVKEQYPGI | (SEQ ID NO:15) |
| A*0201 | 45-53 | AVKEQYPGI | (SEQ ID NO:16) |
| A*0201 | 52-59 | GIEIESRL | (SEQ ID NO:17) |
| A*0201 | 54-62 | EIESRLGGT | (SEQ ID NO:18) |
| A*0201 | 58-67 | RLGGTGAFEI | (SEQ ID NO:19) |
| A*0201 | 61-69 | GTGAFEIEI | (SEQ ID NO:20) |
| A*0201 | 66-73 | EIEINGQL | (SEQ ID NO:21) |
| A*0201 | 66-74 | EIEINGQLV | (SEQ ID NO:22) |
| A*0201 | 88-96 | DLIEAIRRA | (SEQ ID NO:23) |
| A*0201 | 89-96 | LIEAIRRA | (SEQ ID NO:24) |
| A*0201 | 92-101 | AIRRASNGET | (SEQ ID NO:25) |
| A*0201 | 95-102 | RASNGETL | (SEQ ID NO:26) |
| A*0201 | 104-113 | KITNSRPPCV | (SEQ ID NO:27) |
| A*0201 | 105-113 | ITNSRPPCV | (SEQ ID NO:28) |
| A*0201 | 105-114 | ITNSRPPCVI | (SEQ ID NO:29) |
| A*3101 | 16-24 | EVEPGSGVR | (SEQ ID NO:30) |
| B*3501 | 30-38 | EPCGFEATY | (SEQ ID NO:31) |
| A*30101 | 96-104 | ASNGETLEK | (SEQ ID NO:32) |
| supermotif | | | |

*predicted using rules found at the SYFPEITHI website (wysiwyg://35/http://134.2.96.221/scripts/hlaserver.dll/EpPredict.htm) and are based on the book “MHC Ligands and Peptide Motifs” by Rammensee, H. G., Bachmann, J. and S. Stevanovic. Chapman & Hall, New York, 1997.

Non-limiting examples of other peptides derived from cancer cells are described in Table 2.

TABLE 2
Peptides derived from cancer cells which bind to MHC class I

| Peptide Antigen(s) | Expressed in | HLA allele | Ref. |
|---|---|---|---|
| Melan A/MART-1 | Melanoma | A*0201, | 1-3 |
| (26-35) | | | |
| gp 100 (71-78, | Melanoma | A*0201, A11, | 4-6 |
| 280-288) | | A3, Cw8 | |
| Tyrosinase (368-376) | Melanoma | A*0201 | 7 |
| Tyrosinase related | Melanoma | A*0201, A31, | 8-9 |
| protein-2 (180-188, | | A33 (A3 st) | |
| 197-205, 387-395) | | | |
| MAGE-1 (multiple | Melanoma | A1, A2.1, A3.2, | 10 |
| peptides) | | A11, A24 | |
| MAGE-3 (168-176, | Melanoma | A*0101, A*0201 | 11-12 |
| 271-279) | | | |
| MAGE-1, 2, 3, 6 | Melanoma | B*3701 | 13 |
| (127-136) | | | |
| (promiscuous epitope) | | | |
| MC1R melanocyte | Melanoma | A*0201 | 14 |
| stimulating hormone | | | |
| receptor (244, 283, | | | |
| 291) 707-AP | Melanoma | A*0201 | 15 |
| GAGE (1, 2, 3, 4, 5, | Melanoma, others | Cw6 (GAGE1) | 16 |
| 6, 7B, 8) | | | |
| Her2/neu (at least 6 | Breast, ovarian, | A*0201, A3 st | 17-19 |
| epitopes, including | pancreatic, non- | | |
| 654-662, 9(754)) | small cell lung | | |
| | carc., melanoma | | |
| CEA (CAP-1), 9(61) | Colorectal carc., | A3 st, A24 | 17, |
| | others | | 20 |
| Papillomavirus | Cervical squamous | A*0201 | 21-23 |
| type 16 E7 (11-20, | carc. | | |
| 82-90, 86-93) | | | |
| Bcr-abl (4 peptides) | Chronic | A3, A11 | 24-25 |
| | myelogenous | | |
| | leukemia | | |
| p53 (149-157, | Squamous cell | A*0201 | 26 |
| 264-272) | carc. of the head | | |
| | and neck | | |
| RBP-1 (247-256, | Breast carc. | A*0201, A*0301 | 27 |
| 250-259) | | | |

st: supertype

In another embodiment, the peptide is derived from an agent for infectious disease or an infected cell, or stimulates an immune response against an agent for infectious disease. Agents for infectious disease include bacteria, mycobacteria, fingi, worms, protozoa, parasites, viruses, prions, etc. Non-limiting examples of peptides derived from infectious agents are described in Table 3.

TABLE 3
Peptides derived from agents for infectious disease

| Peptide antigen | Expressed in | HLA allele | Ref. |
|---|---|---|---|
| CY1899 (core | Hepatitis B | A*0201 | 28-29 |
| protein 18-27) | | | |
| Nucleocapsid T cell | Hepatitis B | A*0201 | 30 |
| epitope 18-27 | | | |
| NS4.1769 (NS4B, | Chronic hepatitis C | A*0201 | 31-32 |
| NS5B) | | | |
| MN r gp 160 | HIV-1 | A*0201 | 33 |
| Tax (11-19) | HTLV-1 | A*0201 | 34 |
| MP (57-66) | Influenza | A*0201 | 35 |
| SSP2 | Malaria | A*0201, multiple | 36-37 |
| | (Plasmodium | A and B supertypes | |
| | falciparum) | | |
| TSA-1, ASP-1, | Chagas' Disease | A*0201 | 38 |
| ASP-2 | (Trypanosoma | | |
| | cruzi) | | |

Reference List for Tables 3 and 4: 1. Valmori, D. et al., J. Immunol. 161:6956-62 (1998); 2. Brinckerhoff, L. H. et al., Int. J Cancer. 83:326-34 (1999); 3. Rivoltini, L. et al., Cancer Res. 59:301-6 (1999); 4. Castelli, C. et al., J. Immunol. 162:1739-48 (1999); 5. Abdel-Wahab, Z. et al., Cell. Immunol. 186:63-74 (1998); 6. Kawashima, I. et al., Int. J. Cancer. 78:518-24 (1998); 7. Valmori, D. et al., Cancer Res. 59:4050-5 (1999); 8. Parkhurst, M. R. et al., Cancer Res. 58:4895-901 (1998); 9. Wang, R. F. et al., J. Immunol. 160:890-7 (1998); 10. Celis, E. et al., Molecular Immunol. 31:1423-30 (1994); 11. Valmori, D. et al., Cancer Res. 57:735-41 (1997); 12. Fleischhauer, K. et al., J. Immunol. 159:2513-21 (1997); 13. Tanzarella, S. et al., Cancer Res. 59:2668-74 (1999); 14. Salazar-Onfray, F. et al., Cancer Res. 57:4348-55 (1997); 15. Takahashi, T. et al., Clinical Cancer Res. 3:1363-70 (1997); 16. De Backer, O. et al., Cancer Res. 59:3157-65 (1999); 17. Kawashima, I. et al., Cancer Res. 59:431-5 (1999); 18. Kono, K. et al., Int. J. Cancer. 78:202-8 (1998); 19. Peiper, M. et al., Anticancer Res. 19:2471-5 (1999); 20. Nukaya, I. et al., Int. J. Cancer. 80:92-7 (1999); 21. Steller, M. A. et al., Clin. Cancer Res. 4:2103-9 (1998); 22. Alexander, M. et al., Am. J. Obstetrics and Gynecology 175:1586-93 (1996); 23. Ressing, M. E. et al., J. Immunol. 154:5934-43 (1995); 24. Bocchia, M. et al., Blood 87:3587-92 (1996); 25. Bocchia, M. et al., Blood 85:2680-4 (1995); 26. Chikamatsu, K. et al., Clinical Cancer Res. 5:1281-8 (1999); 27. Takahashi, T. et al., Br. J. Cancer 81:342-9 (1999); 28. Heathcote, J. et al., Hepatology 30:531-6 (1999); 29. Livingston, B. D. et al., J. Immunol 159:1383-92 (1997); 30. Bertoletti, A. et al., Hepatology 26:1027-34 (1997); 31. Alexander J. et al., Human Immunol. 59:776-82 (1998); 32. Battegay, M. et al., J. Virol. 69:2462-70 (1995); 33. Kundu, S.K. et al., AIDS Research and Human Retroviruses 14:1669-78 (1998); 34. Hollsberg, P. et al., Proc. Natl. Acad. Sci. USA. 92:4036-40 (1995); 35. Gotch, F. et al., Nature 326:881-2 (1987); 36. Doolan, D. L. et al., Immunity 7:97-112 (1997);

37. Wizel, B. et al., J. Immunol. 155:766-75 (1995); and 38. Wizel, B. et al., J. Clin. Invest. 102:1062-71 (1998).

The antigenic peptide may also be derived from a target tissue from autoimmune disease or from an allergen. Compounds comprising these antigenic peptides which suppress an immune response are especially preferred.

Further, the antigenic peptide may be synthetic. The synthetic peptide may provoke an immune response against cancerous cells or virus-infected cells. Alternatively, the synthetic peptide may downregulate an undesirable immune response, e.g, autoimmunity or allergy.

The sequence of antigenic peptide epitopes known to bind to specific MHC molecules can be modified at the known peptide anchor positions in predictable ways that act to increase MHC binding affinity. Such “epitope enhancement” has been employed to improve the immunogenicity of a number of different MHC class I binding peptide epitopes (Berzofsky, J. A. et al., Immunol. Rev. 170:151-72 (1999); Ahlers, J. D. et al., Proc. Natl. Acad. Sci U.S.A. 94:10856-61 (1997); Overwijk, et al., J. Exp. Med. 188:277-86 (1998); Parkhurst, M. R. et al., J. Immunol. 157:2539-48 (1996)).

Antibodies are constructed of one, or several, units, each of which consists of two heavy (H) polypeptide chains and two light (L) polypeptide chains. The H and L chains are made up of a series of domains. The L chains, of which there are two major types (θ and λ), consists of two domains. The H chains are of several types, including μ, δ, and γ (of which there are several subclasses), α and ε. In humans, there are eight genetically and structurally identified antibody classes and subclasses as defined by heavy chain isotypes: IgM, IgD, IgG3, IgG1, IgG2, IgG4, IgE, and IgA. Further, for example, “IgG” means an antibody of the G class, and that, “IgG1” refers to an IgG molecules of subclass 1 of the G class. IgG1 antibodies, like all antibodies of the IgG class, are comprised of 4 domains, one of which is variable and the other 3 are constant. An Fab antibody fragment is comprised of an intact light chain and a truncated heavy chain that each comprise two domains, one variable and one constant. The MHC class I α chain is comprised of 3 domains and β₂-microglobulin of 1 domain each with broad structural similarities to antibody domains. In this respect, both MHC class I α chain and β₂-microglobulin are classified as members of the immunoglobulin gene superfamily. The structural conservation reflected in these domains may facilitate synthesis, expression and manufacture of the antibody or antibody fragment fusion proteins comprised in the compounds of this invention. For example, fusion of two MHC class I α3 domains to the carboxyl terminus of the truncated heavy chain of an Fab antibody fragment would reconstitute a 4 domain molecule of about the same size as a complete heavy chain. Proper folding and stability of this chain may be favored by interaction with β2-microglobulin.

As used herein, the term “antibody” (Ab) or “monoclonal antibody” (MAb) is meant to include intact molecules as well as antibody portions (such as, for example, Fab and F(ab′)₂ portions and Fv fragments) which are capable of specifically binding to a cell surface marker. Such portions are typically produced by proteolytic cleavage, using enzymes such as papain (to produce Fab portions) or pepsin (to produce F(ab′)₂ portions). Especially preferred in the compounds of the invention are Fab portions. Alternatively, antigen-binding portions can be produced through the application of recombinant DNA technology.

The immunoglobulin can be a “chimeric antibody” as that term is recognized in the art. Also, the immunoglobulin may be a “bifunctional” or “hybrid” antibody, that is, an antibody which may have one arm having a specificity for one antigenic site, such as a tumor associated antigen while the other arm recognizes a different target, for example, an immunologically active cytokine or lymphokine, an MHC class I α3 domain or a hapten which is bound to an immunologically active protein or an MHC class I α3 domain. In any case, the hybrid antibodies have a dual specificity, preferably with one or more binding sites specific for an antigen expressed on the surface of a target cell, for example, an antigen associated with a tumor, an infectious organism, or antigenic marker of another disease state.

Biological bifunctional antibodies are described, for example, in European Patent Publication, EPA 0 105 360, to which those skilled in the art are referred. Such hybrid or bifunctional antibodies may be derived, as noted, either biologically, by cell fusion techniques, or chemically, especially with cross-linking agents or disulfide bridge-forming reagents, and may be comprised of whole antibodies and/or fragments thereof. Methods for obtaining such hybrid antibodies are disclosed, for example, in PCT application WO83/03679, published Oct. 27, 1983, and published European Application EPA 0 217 577, published Apr. 8, 1987. Particularly preferred bifunctional antibodies are those biologically prepared from a “polydome” or “quadroma” or which are synthetically prepared with cross-linking agents such as bis-(maleimideo)-methyl ether (“BMME”), or with other cross-linking agents familiar to those skilled in the art.

In addition the immunoglobin may be a single chain antibody (“SCA”). These may consist of single chain Fv fragments (“scFv”) in which the variable light (“V[L]”) and variable heavy (“V[H]”) domains are linked by a peptide bridge or by disulfide bonds. Also, the immunoglobulin may consist of single V[H ]domains (dAbs) which possess antigen-binding activity. See, e.g., G. Winter and C. Milstein, Nature 349:295 (1991); R. Glockshuber et al., Biochemistry 29:1362 (1990); and, E. S. Ward et al., Nature 341:544 (1989).

Also preferred for use in the present invention are chimeric monoclonal antibodies, preferably those chimeric antibodies having specificity toward a tumor associated antigen or an antigen of a pathogen infected cell. As used in this example, the term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Other forms of chimeric antibodies encompassed by the invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art. See, e.g., Morrison, S. L. et al., Proc. Nat'l Acad. Sci. 81:6851 (1984).

Encompassed by the term “chimeric antibody” is the concept of “humanized antibody”, that is those antibodies in which the framework or “complementarity” determining regions (“CDR”) have been modified to comprise the CDR of an immunoglobulin of different specificity as compared to that of the parent immunoglobulin. In a preferred embodiment, a murine CDR is grafted into the framework region of a human antibody to prepare the “humanized antibody”. See, e.g., L. Riechmann et al., Nature 332:323 (1988); M. S. Neuberger et al., Nature 314:268 (1985). Particularly preferred CDR'S correspond to those representing sequences recognizing the antigens noted above for the chimeric and bifunctional antibodies. The reader is referred to the teaching of EPA 0 239 400 (published Sep. 30, 1987), for its teaching of CDR modified antibodies.

Most preferably, fully human antibodies or fragments thereof are used in the compounds of the invention, preferably those fully human antibodies having specificity toward a tumor associated antigen or an antigen of a pathogen infected cell. Methods have been described for selection of fully human antibodies in human immunoglobulin transgenic mice, from libraries of human immunoglobulin genes constructed in phage and expressed in bacteria or constructed in a mammalian viral expression vector for expression in mammalian cells, and from human hybridoma cells.

One skilled in the art will recognize that a bifunctional-chimeric antibody can be prepared which would have the benefits of lower immunogenicity of the chimeric, humanized or fully human antibody, as well as the flexibility, especially for therapeutic treatment, of the bifunctional antibodies described above. Such bifunctional-chimeric antibodies can be synthesized, for instance, by chemical synthesis using cross-linking agents and/or recombinant methods of the type described above. In any event, the present invention should not be construed as limited in scope by any particular method of production of an antibody whether bifunctional, chimeric, bifunctional-chimeric, humanized, fully human or an antigen-recognizing fragment or derivative thereof.

In addition, the invention encompasses within its scope immunoglobulins (as defined above) or immunoglobulin fragments to which are fused active proteins, for example, an enzyme of the type disclosed in Neuberger et al., PCT application, WO86/01533, published Mar. 13, 1986. The disclosure of such products is incorporated herein by reference.

As noted, “bifunctional”, “fused”, “chimeric” (including humanized), “fully human”, and “bifunctional-chimeric” (including humanized) or “bifunctional-fully human” antibody constructions also include, within their individual contexts constructions comprising antigen recognizing fragments. As one skilled in the art will recognize, such fragments could be prepared by traditional enzymatic cleavage of intact bifunctional, chimeric, humanized, fully human or chimeric-bifunctional or fully human-bifunctional antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin “fragment”. It is in this context, therefore, that the term “fragment” is used.

Furthermore, as noted above, the immunoglobulin (antibody), or fragment thereof, used in the present invention may be polyclonal or monoclonal in nature. Monoclonal antibodies are the preferred immunoglobulins, however. The preparation of such polyclonal or monoclonal antibodies now is well known to those skilled in the art who, of course, are fully capable of producing useful immunoglobulins which can be used in the invention. See, e.g., G. Kohler and C. Milstein, Nature 256:495 (1975). In addition, hybridomas and/or monoclonal antibodies which are produced by such hybridomas and which are useful in the practice of the present invention are publicly available from sources such as the American Type Culture Collection (“ATCC”) 10801 University Boulevard, Manassas, Va. 20110-2209 or, commercially, for example, from Boehringer-Mannheim Biochemicals, P.O. Box 50816, Indianapolis, Ind. 46250.

The antibodies of the present invention may be prepared by any of a variety of methods. For example, cells expressing the cell surface marker or an antigenic portion thereof can be administered to an animal in order to induce the production of sera containing polyclonal antibodies. In a preferred method, a preparation of protein is prepared and purified as to render it substantially free of natural contaminants. Such a preparation is then introduced into an animal in order to produce polyclonal antisera of greater specific activity.

In the most preferred method, the antibodies of the present invention are monoclonal antibodies (or portions thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., pp. 563-681 (1981)). In general, such procedures involve immunizing an animal (preferably a mouse) with a protein antigen or, more preferably, with a protein-expressing cell. Suitable cells can be recognized by their capacity to bind antibody. Such cells may be cultured in any suitable tissue culture medium; however, it is preferable to culture cells in Excell hybridoma medium (JRH Biosciences, Lenexa, Kans.) with 5% fetal bovine serum. The splenocytes of such immunized mice are extracted and fused with a suitable myeloma cell line. Any suitable myeloma cell line may be employed in accordance with the present invention; however, it is preferable to employ the parent myeloma cell line (SP₂O), available from the American Type Culture Collection, 10801 University Boulevard, Manassas, Va. 20110-2209. After fusion, the resulting hybridoma cells are selectively maintained in HAT medium, and then cloned by limiting dilution as described by Wands et al., Gastroenterology 80:225-232 (1981). The hybridoma cells obtained through such a selection are then assayed to identify clones which secrete antibodies capable of binding the antigen.

It malt be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

It is most preferable to use fully human antibodies. A method for selection of fully human antibodies from libraries of human immunoglobulin genes constructed in vaccinia virus is described in Zauderer, M. et al. WO 01/72995, published 4 Oct. 2001, the disclosure of which is incorporated by reference herein.

The compounds of the present invention may be labeled, so as to be directly detectable, or will be used in conjunction with secondary labeled immunoreagents which will specifically bind the compound for example, for detection or diagnostic purposes. Labels of interest may include dyes, enzymes, chemiluminescers, particles, radioisotopes, or other directly or indirectly detectable agent. Alternatively, a second stage label may be used, e.g. labeled antibody directed to one of the constituents of the compound of the invention.

Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹n, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ^{47Sc}, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radio nucleotide has a more favorable gamma emission energy for imaging (Perkins et al., Eur. J. Nucl. Med. 10:296-301 (1985); Carasquillo et al., J. Nucl. Med. 28:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and Fe.

Typical techniques for binding the above-described labels to antibodies are provided by Kennedy et al., Clin. Chim. Acta 70:1-31 (1976), and Schurs et al., Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

In one embodiment, the antibody is specific for a cell surface marker of a professional antigen presenting cell. Preferably, the antibody is specific for a cell surface marker of a dendritic cell, for example, CD83, CMRF-44, CMRF-56, or DEC205 and BDCA-2, BDCA-3, or BDCA-4 as described by Dzionek A et al. J. Immunol. 165:6037-6046(2000). The antibody may be specific for a cell surface marker of another professional antigen presenting cell, such as a B cell or a macrophage. CD20 is expressed on B cells, and other markers have been described for other antigen presenting cells.

In another embodiment, the antibody is specific for a cell surface marker of a T cell, for example, CD28, CTLA-4 (CD 152), or CD25. The combination of TCR mediated signal from the peptide-MHC complexes (signal 1) and co-stimulator signal through CD28 (signal 2) results in strong T cell stimulation. In contrast, the combination of TCR mediated signal from the peptide-MHC complexes (signal 1) and co-stimulator signal through CTLA-4 results in the inhibition of previously activated T cells or stimulation of antigen-specific inhibitors of activation of other T cells and may be especially useful for amelioration of autoimmune responses. CD25 is an IL-2 receptor upregulated upon T cell activation. Anti-CD25 fusion proteins could, therefore, specifically target T cells in an activated state. In any application targeting T cells, the antigen presenting cell expressing peptide-MHC complexes may be another MHC class I positive T cell or any antigen presenting cell in the microenvironment.

CTLA-4 is a molecule expressed by activated T lymphocytes with very high affinity for costimulatory molecules B7-1 and B7-2 and has been reported to mediate signals that dampen or downregulate immune responsiveness (Bluestone, J. A., J. Immunol. 158:1989 (1997)). Although in most murine studies CTLA-4 specific antibodies have been reported to act antagonistically to block inhibitory effects, some human CTLA-4 specific monoclonal antibodies have been described that inhibit responses of resting human CD4+ T cells (Blair, P. J. et al., J. Immunol. 160:12-15 (1998)). The mechanisms of inhibition have not been fully characterized and may be mediated by either or both a direct inhibitory effect on T cells that have upregulated expression of CTLA-4 or through activation of a subset of inhibitory T cells that express high levels of CTLA-4. In either case, simultaneous binding of CTLA-4 and T cell receptor on a T cell by a CTLA-4 specific antibody and interaction with peptide-MHC may result in the inhibition of undesirable T cell reactivity for that peptide.

T and B lymphocytes express a variety of surface molecules that, when crosslinked by antibodies, induce positive or negative signals that culminate in responsiveness or unresponsiveness. For the purpose of antigen delivery to T and B cells, it may, in some cases, be inadvisable to crosslink a cell surface antigen with divalent or polyvalent antibody since this may induce massive cell proliferation and splenomegaly in vivo (e.g. crosslinking CD3 or CD28 on T cells, or CD40 on B cells with specific antibody) or widespread cell death (anti-Fas antibody kills mice within hours of injection). Rather, it would be desirable simply to dock an MHC class I α3 complex on the lymphocyte surface using compounds of the invention with only monovalent antibody specificity. These targeted MHC class I α3 complexes can be employed to induce proliferation or cytotoxic activity of peptide-specific T lymphocytes either in vitro or in vivo.

In another embodiment, the antibody is specific for a cell surface marker of a non-immune cell, for example, a tumor cell. Tumors evade the immune system in multiple ways, including downregulation of some subgroups of MHC proteins on the surface or interference with mechanisms of antigen processing. The compounds of the invention that specifically target tumor cells by virtue of antibody specific for antigens present on the tumor cell surface will increase presentation of peptides available for specific T cell recognition and activation.

In other embodiments, the antibody is specific for a cell surface marker of an infected cell such as the membrane proteins of an enveloped virus.

Epithelial cells and fibroblasts are non-professional antigen presenting cells. Although they express MHC class I molecules and can be induced to express MHC class II after exposure to IFN-gamma, they are not fully competent to stimulate naïve T cells because they fail to express costimulatory molecules such as B7-1 and B7-2. Indeed, a signal through the T cell antigen receptor alone in the absence of a second costimulatory signal induces tolerance in naive T cells. By targeting compounds of the invention to these non-professional antigen presenting cells, it should be possible to effectively induce tolerance to the immunodominant peptide:MHC complexes of interest. A commercially available antibody, Ber-EP4 (Latza, U. et al., J. Clin. Pathol. 43:213-9 (1990), DAKO), reacts with two glycoproteins expressed on the surface of all epithelial cells except superficial squamous epithelial cells, hepatocytes, and parietal cells and has similar reactivity to HEA 125 (Moldenhauer, G. et al., Br. J. Cancer. 56:714-21 (1987)). Fibroblast-specific surface markers and antibodies that target them are under investigation in numerous laboratories and one potential candidate has been identified (Fearns, C and Dowdle, E B. Int. J Cancer. 50:621-7 (1992), Miltenyi Biotech) that could be similarly employed to promote T cell unresponsiveness to particular peptides. Alternatively, in those embodiments of the invention that incorporate B7 linked to β2-microglobulin, the deficiency of costimulator activity in epithelial cells or fibroblasts could be complemented for effective T cell stimulation.

It has been reported that the liver is a site of accumulation of activated T lymphocytes about to undergo activation induced cell death (AICD) and that sinusoidal endothelial cells and Kupffer cells may constitute a “killing field” for activated CD8⁺ T cells originating from peripherel lymphoid organs (Mehal, Juedes and Crispe, J. Immunol. 163:3202-3210 (1999); Crispe, I. N. Immunol. Res. 19:143-57 (1999)). Compounds of the invention can promote trapping and deletion of specific T cells in the liver by targeting specific peptide:MHC complexes to the liver with anti-hepatocyte specific antibodies.

In a preferred embodiment, the immune system's extraordinary power to eradicate pathogens is redirected to target an othenvise evasive tumor. The immune response to commonly encountered pathogens (eg influenza virus) and/or pathogens or toxins against which individuals are likely to have been vaccinated (eg influenza, or tetanus) is associated with induction of a high frequency of high avidity T cells that are specific for immunodominant peptide:MHC complexes of cells infected with these pathogens. These same highly represented, high avidity T cells can be redirected to tumors by linking the appropriate peptide recognized by these T cells to a tumor-specific antibody specificity.

Non-limiting examples of cell surface markers appropriate for immune targeting of the compounds of the present invention are presented in Tables 4 and 5.

TABLE 4
Human leukocyte differentiation antigens

| Surface Antigen | Expressed by | Ref. |
|---|---|---|
| CD2 | T lymphocytes | 1-2 |
| CD4 | T cell subset | 1 |
| CD5 | T lymphocytes | 1 |
| CD6 | T lymphocytes | 1, 3 |
| CD8 | T cell subset | 1 |
| CD27 | Naive CD4 T cell subset | 4 |
| CD31 | Naive CD4 T cell subset | 4 |
| CD25 | Activated T cells | 1 |
| CD69 | Activated T cells | 1, 5, 6 |
| HLA-DR | Activated T cells, APC | 7 |
| CD28 | T lymphocytes | 8 |
| CD152 (CTLA-4) | Activated T cells | 9 |
| CD154 (CD40L) | Activated T cells | 10 |
| CD19 | B lymphocytes | 1, 11 |
| CD20 | B lymphocytes | 1 |
| CD21 | B lymphocytes | 1 |
| CD40 | Antigen presenting cells | 12-13 |
| CD134 (OX40) | Antigen presenting cells | 13-14 |
| B7-1 and 2 | Antigen presenting cells | 13, 15, 16 |
| CD45 | Leukocytes | 1 |
| CD83 | Mature dendritic cells | 17 |
| CMRF-44 | Mature dendritic cells | 18 |
| CMRF-56 | Mature dendritic cells | 19 |
| OX40L | Dendritic cells | 20 |
| DEC-205 | Dendritic cells | 21 |
| TRANCE/RANK | Dendritic cells | 22 |
| receptor | | |

Reference listing for table 4:1. Knapp, W. et al., eds., Leukocyte Typing IV: White Cell Differentiation Antigens, Oxford University Press, New York. (1989); 2. Bierer, B. E. et al., Seminars in Immunology. 5:249-61 (1993); 3. Rasmussen, R. A. et al., J. Immunol. 152:527 (1994); 4. Morimoto, C. et al., Clin. Exp. Immunol. 11:241-7 (1993); 5. Ziegler, S. F. et al., Stem Cells 12:456-65 (1994); 6. Marzio, R. et al., CD69 and regulation of immune function. 21:565-82 (1999); 7. Rea, I. M. et al., Exp. Gerontol. 34:79-93 (1999); 8. June, C. H. et al., Immunology Today 11:211 (1993); 9. Lindsten, T. et al., J. Immunol. 151:3489 (1993); 10. Mackey, M. F. et al, J. Leukocyte Biol. 63:418-28 (1998); 11. Bradbury, L. E. et al, J. Immunol. 151:2915 (1993); 12. Clark, E. A., and Ledbetter, J. A., Proc. Natl. Adad. Sci. USA. 83:4494 (1986); 13. Schlossman, S. et al., eds. Leukocyte Typing V: White Cell Differentiation Antigens. Oxford University Press, New York (1995); 14. Latza, U. et al., Eur. J. Immunol. 24:677 (1994); 15. Koulova, L. et al., J Exp. Med. 173:759 (1991); 16. Azuma, M. et al., Nature 366:76 (1993); 17. Zhou, L. J., and Tedder, T.F., J. Immunol. 154: 3821 (1995); 18. Vuckovic, S. et al., Exp. Hematolagy 26:1255 (1998); 19. Hock, B. D. et al., Tissue Antigens 53:320-34 (1999); 20. Chen, A. I. et al., Immunity 11:689 (1999); 21. Kato, M. et al, Immunogenetics. 47:442 (1998); and 22. Anderson, D. M. et al., Nature 390:175 (1997).

TABLE 5
Tumor cell surface antigens recognized by antibodies

| Antigen(s) | Expressed in | Ref. |
|---|---|---|
| CEA | Colorectal, thyroid carcinoma, others | 1-6 |
| Her2/neu | Breast, ovarian carcinomas | 7 |
| CM-1 | Breast | 8 |
| MUC-1 | Pancreatic carcinoma, others | 9-10 |
| 28K29 | Lung adenocarcinoma, large cell carcinoma | 11 |
| E48 | Head and neck squamous cell carcinoma | 12 |
| U36 | Head and neck squamous cell carcinoma | 12 |
| NY-ESO-1* | Esophageal carcinoma, melanoma, others | 13-14 |
| KU-BL 1-5* | Bladder carcinoma | 15 |
| NY CO 1-48* | Colon carcinoma | 16 |
| HOM MEL 40* | Melanoma | 17 |
| OV569 | Ovarian carcinoma | 18 |
| ChCE7 | Neuroblastoma, renal cell carcinoma | 19 |
| CA19-9 | Colon carcinoma | 20 |
| CA125 | Ovarian carcinoma | 21 |
| Gangliosides | Melanoma, neuroblastoma, others | 22 |
| (GM2, GD2, 9-o- | | |
| acetyl-GD3, | | |
| GD3) | | |

*Antigens identified using SEREX technology.

Reference List for Table 5: 1. Juweid, M. E. et al., Cancer 85:1828-42 (1999); 2. Stewart, L. M. et al., Imunotherapy 47:299-306 (1999); 3. Robert, B. et al., International J. Cancer 81:285-91 (1999); 4. Kraeber-Bodere, F. et al., J. Nuclear Medicine 40:198-204 (1999); 5. Kawashima, I. et al., Cancer Res. 59:431-5 (1999); 6. Nasu, T. et al., Immunology Letters 67:57-62 (1999); 7. Zhang, H. et al., Experimental & Molecular Pathology 67:15-25 (1999); 8. Chen, L. et al., Acta Academiae Medicinae Sinicae 19(2):150-3; 9. Beum, P. V. et al., J. Biol. Chem. 274:24621-8 (1999); 10. Koumarianou A. A. et al., British J. Cancer 81:431-9 (1999); 11. Yoshinari, K. et al., Lung Cancer 25:95-103 (1999); 12. Van Dongen, G. A. M. S. et al., Anticancer Res. 16:2409-14 (1996); 13. Jager, E. et al., J. Exp. Med. 187:265-70 (1998); 14. Jager, E. et al., International J. Cancer 84:506-10 (1999); 15. Ito, K. et al., AUA 2000 Annual Meeting, Abstract 3291 (2000); 16. Scanlan, M. J. et al., International J. Cancer 76:652-8 (1998); 17. Tureci, O. et aL, Cancer Res. 56:4766-72 (1996); 18. Scholler, N. et al., Proc. Natl. Acad, Sci. USA 96:11531-6 (1999); 19. Meli, M. L. et al., International J. Cancer 83:401-8 (1999); 20. Han, J. S. et al., Cancer 76:195-200 (1995); 21. O'Brien, T. J. et al., International J. Biological Markers 13:188-95 (1998); and 22. Zhang, S. et al., Cancer Immunol. Immunotherapy 40:88-94 (1995).

The conjugation of the MHC class I α3 complex(es) to the antibody may be conducted in any suitable manner. For example, the coupling may be of a physical and/or chemical type. The antibody and MHC class I α3 complex may be coupled physically utilizing a carrier for example a Sepharose carrier (available from Pharmacia, Uppsala, Sweden) or recently developed microsphere technology. (Southern Research Institute).

Alternatively, the MHC class I α3 domain and the antibodies may be linked together directly. A number of reagents capable of cross-linking proteins are known in the art, illustrative entities include: azidobenzoyl hydrazide, N-[4-(p-azidosalicylamino)butyl]-3′-[2′-pyridyldithio] propionamide), bis-sulfosuccinimidyl suberate, dimethyladipimidate, disuαinimidyltartrate, N-γ-maleimidobutyryloxysuαinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, formaldehyde and succinimidyl 4-[N-maleimidomethyl] cyclohexane-1-carboxylate.

Alternatively, the MHC class I α3 complex can be genetically modified by including sequences encoding amino acid residues with chemically reactive side chains such as Cys or His. Such amino acids with chemically reactive side chains may be positioned in a variety of positions of the MHC class I α3 domain, preferably distal to the site where β2-microglobulin and MHC Class I α3 interact. Suitable side chains can be used to chemically link two or more MHC class I α3 complexes to a suitable dendrimer particle. Dendrimers are synthetic chemical polymers that can have any one of a number of different functional groups on their surface (D. Tomalia, Aldrichimica Acta 26:91:101 (1993)). Exemplary dendrimers for use in accordance with the present invention include e.g. E9 starburst polyamine dendrimer and E9 combburst polyamine dendrimer, which can link cysteine residues.

A short linker amino acid sequence may be inserted between the MHC class I α3 complex(es) and the antibody. The length of the linker sequence will vary depending upon the desired flexibility to regulate the degree of antigen binding and cross-linking. If a linker sequence is included, this sequence will preferably contain at least 3 and not more than 30 amino acids. More preferably, the linker is about 5, 10, 15, 20, or 25 amino acids long. Generally, the linker consists of short glycine/serine spacers, but any known amino acid may be used. Examples of linkers known to those skilled in the art include (Gly₄Ser)₃ (SEQ ID NO:33)and (Gly₄Ser)₂Gly₃AlaSer (SEQ ID NO:34).

Fusion of MHC molecules to the amino terminus of the immunoglobulin chain variable regions has been previously described (Dal Porto, J. et al., Proc. Natl. Acad. Sci., USA 90:6671-75 (1993)). Although this fusion product does not interfere with recognition of haptens in fusion products with hapten-specific antibody, the proximity of MHC class I α3 complex and antibody binding site makes it more likely that the MHC class I α3 complex could interfere with antibody binding to macromolecular determinants embedded in a complex membrane. There is, therefore, a need for new compounds that can serve the requirements of targeted delivery of MHC cornplexes to T cells and their antigen-specific receptor. Localization of the MHC class I αdomain at the carboxyl terminus of immunoglobulin chains serves this purpose. The MHC complex is well separated from the antibody binding site and is unlikely to interfere with its targeting specificity.

MHC molecules fused to the carboxyl terminus of the exceptionally long IgG3 hinge region or to the CH3 domain, are especially far removed from possible interference with the antigen binding site or its ligand. Fc binding function is preserved in the compounds of this invention that are based on CH3 fusions. It is possible that this would extend the half-life of these compounds in vivo.

There are several other ways to assemble polymeric MHC class I α3 domains on a targeting antibody besides direct antibody-MHC class I α3 domain fusion or the binding of biotinylated MHC class I α3 domains to antibody-avidin fusion proteins. Cochran, J. R. et al., Immunity 12:241-50 (2000) describe the use of chemically synthesized peptide-based cross-linking reagents in which two or more thiol-reactive maleimide groups are linked to lysine side chains in a flexible peptide of 8 to 19 residues containing glycine, serine, and glutamic acid in addition to the modified lysine residues. The MHC class I α3 chain is modified to introduce a cysteine residue at the carboxyl terminus. Following synthesis in E. coli, a complete cysteine modified MHC class I α3 complex is assembled in vitro in the presence of β2-microglobulin. Cysteine modified MHC class I α3 domains react with the maleimide groups on the various peptide backbones with either two, three, or four modified lysine residues for formation of MHC class I α3 domain dimers, trimers, and tetramers. In a preferred embodiment, a carboxyl terminal cysteine modified immunoglobulin chain or fragment thereof could also be synthesized for reaction with a maleimide-modified lysine residue on the same backbone peptide and at the same time as the cysteine modified MHC class I α3 domains.

The alternative embodiments of this invention, direct fusion of antibody and MHC class I α3 domains or indirect association of antibody and MHC class I α3 complexes through a multivalent entity, are respectively advantageous in different situations. The direct fusion simplifies production of the compound while the multivalent entity, as indicated above, can present a larger number of more diverse ligands. In both cases it is desirable to design products that induce minimal immune reactivity. In the case of direct immunoglobulin-MHC class I α3 domain fusion proteins, this is accomplished by employing species compatible antibodies and MHC class I α3 domains joined by simple linkers with a relatively non-immunogenic composition. Multivalent entities may be similarly selected to minimize immunogenicity. Chicken avidin is thought to be relatively nonimmunogenic because of its high concentration in egg products and the well-known propensity of oral infusion to induce immune tolerance (Shin, S. U. et al., J. Immunology 158: 4797-4804 (1997)). It may, in addition, be possible to develop protocols, including some that employ compounds of this invention, that induce specific tolerance.

The attachment site on the MHC class I α3 complex or antibody for binding to a multivalent compound may be naturally occurring, or may be introduced through genetic engineering. The site will be a specific binding pair member or one that is modified to provide a specific binding pair member, where the complementary pair has a multiplicity of specific binding sites. Binding to the complementary binding member can be a chemical reaction, epitope-receptor binding or hapten-receptor binding where a hapten is linked to the subunit chain.

In a preferred embodiment, the MHC class I α chain contains an amino acid sequence which is a recognition site for a modifying enzyme. Preferably, the recognition site is near the carboxyl terminus of the MHC class I α3 domain. Modifying enzymes include BirA, various glycosylases, farnesyl protein transferase, and protein kinases. The group introduced by the modifying enzyme, e.g. biotin, sugar, phosphate, famesyl, etc. provides a complementary binding pair member, or a unique site for further modification, such as chemical cross-linking, biotinylation, etc. that will provide a complementary binding pair member.

For example, the MHC class I α3 domain may be engineered to contain a site for biotinylation, for example a BirA-dependent site. The antibody or fragment thereof can be linked to avidin either directly or indirectly. Direct linkage is accomplished by making an antibody-avidin fusion protein through genetic engineering as described in, for example, Shin et al., Shin, S.-U. et al., J. Immunol. 158:4797-4804 (1997); and Penichet et al., J. Immunol. 163:4421-4426.

In another embodiment, indirect linkage can be effected by employing the previously described construct incorporating genes for the heavy and light chain variable regions of an antibody specific for the hapten dansyl (Shin, S.-U. et al., J. Immunol. 158:4797-4804 (1997)). MHC complexes assembled on the anti dansyl-avidin fusion protein could then associate with any dansylated antibody with the desired targeting specificity. Dansyl chloride (DNS, Molecular Probes cat #D21, 5-dimethylaminonapthalene-1-sulfonyl chloride) is freshly dissolved in dimethyl formamide, 0.1-1 mg/ml. DNS solution (1 μl) is added to 10 μl (20 μg) of purified antibody (2 mg/ml) dissolved in 0.1M NaHCO₃. After one hour incubation at 4° C. with rotation, the reaction is quenched with 2 μl of 0.1M glycine. For each antibody, it is necessary to titrate the DNS concentration to empirically determine the amount necessary to label the antibody while still retaining antibody specificity.

In one embodiment, the compound of the invention incorporates an antibody specificity for a particular immunoglobulin class or isotype, in a preferred embodiment this is an IgG isotype whose expression is regulated by cytokines secreted by Th1 type T cells. Compounds of the invention with this immunoglobulin isotype specificity will bind antigen-specific humoral antibodies of this isotype. The bound humoral antibody will, as a result, target the linked MHC class I α3 complex and any linked cytokines to those cells that express the specific foreign antigens or autoantigens that were responsible for inducing this specific antibody response. The rationale is that, without prior knowledge of the specific antigens targeted in this cancer or infectious disease, it will be possible to deliver desired markers or signals to eradicate the cellular source of specific antigen on IgG antibody coated target cells.

The present invention also relates to vectors which include a nucleotide sequence encoding a compound of the present invention or parts thereof, host cells which are genetically engineered with the recombinant vectors, and the production of the compounds of the present invention or parts thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, or, in mammalian cells, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art. For example, MHC class I molecules can be expressed in Drosophila cells (U.S. Pat. No. 6,001,365).

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others; are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved phar-macokinetic properties (EP-A 0232 262). On the other hand, for some uses it would be desirable to be able to delete the Fc part after the fusion protein has been expressed, detected and purified in the advantageous manner described. This is the case when the Fc portion proves to be a hindrance to use in therapy and diagnosis, for example when the fusion protein is to be used as an antigen for immunizations. In drug discovery, for example, human proteins, such as the hIL5-receptor, have been fused with Fc portions for the purpose of high-throughput screening assays to identify antagonists of hIL-5. See, D. Bennett et al., J. Mol. Recognition 8:52-58 (1995) and K. Johanson et al., J. of Biol. Chem. 270(16):9459-9471 (1995).

Several reports have described secretion and assembly of fusion proteins comprised of diverse sequences linked to the carboxyl terminus of immunoglobulin chains (Harvill, E. T. et al., J. Immunol. 157:3165-70 (1996); Shin, S. U. et al., J. Imnmunology 158: 4797-4804 (1997); Penichet, M. L. et al., J. Immunol. 163:4421-26 (1999); Zhang, H. F. et al., J. Clin. Invest 103:55-61 (1999)). Fusion proteins of the compounds of this invention will likewise retain anino terminal sequences of the immunoglobulin chain that direct secretion. MHC class I α3 domains linked to the carboxyl terminus of the immunoglobulin chains are stripped of hydrophobic transmembrane sequences and should not interfere with secretion.

The polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides useful in the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

The ability of a compound of the present invention to modulate an immune resonse can be readily determined by an in vitro assay. T cells for use in the assays include transformed T cell lines, such as T cell hybridomas, or T cells which are isolated from a mammal, e.g., from a human or from a rodent such as a mouse. T cells can be isolated from a mammal by known methods.

See, for example, Shimonkevitz et al., J. Exp. Med. 158:303 (1983).

A suitable assay to determine if a compound of the present invention is capable of modulating the activity of T cells is conducted by coculturing T cells and antigen presenting cells, adding the particular compound of interest to the culture medium, and measuring IL-2 production. A decrease in IL-2 production over a standard indicates the compound can suppress an immune response. An increase in IL-2 production over a standard indicates the compound can stimulate an immune response.

The T cells employed in the assays are incubated under conditions suitable for proliferation. For example, a DO11.10 T cell hybridoma is suitably incubated at about 37° C. and 5% CO₂ in complete culture medium (RPMI 1640 supplemented with 10% FBS, penicillin/streptomycin, L-glutamine and 5×10⁻⁵ M 2-mercaptoethanol). Serial dilutions of the compound can be added to the T cell culture medium. Suitable concentrations of the compound added to the T cells typically will be in the range of from 10¹² to 10⁶ M. Use of antigen dose and APC numbers giving slightly submaximal T cell activation is preferred to detect stimulation or inhibition of T cell responses by the compounds of the invention.

Alternatively, rather than measurement of an expressed protein such as IL-2, modulation of T cell activation can be suitably determined by changes in antigen-dependent T cell proliferation as measured by radiolabelling techniques as are recognized in the art. For example, a labeled (e.g., tritiated) nucleotide may be introduced to an assay culture medium. Incorporation of such a tagged nucleotide into DNA serves as a measure of T cell proliferation. This assay is not suitable for T cells that do not require antigen presentation for growth, e.g., T cell hybridomas. A difference in the level of T cell proliferation following contact with the compound of the invention indicates the complex modulates activity of the T cells. For example, a decrease in T cell proliferation indicates the compound can suppress an immune response. An increase in T cell proliferation indicates the compound can stimulate an immune response.

Additionally, the ⁵¹Cr release assay, described below, can be used to determine CTL activity.

These in vitro assays can be employed to select and identify peptide containing MHC class I α3 complexes that are capable of modulating an immune response. Assays described above, e.g., measurement of IL-2 production or T cell proliferation, are employed to determine if contact with the compound modulates T cell activation.

In vivo assays also may be suitably employed to determine the ability of a compound of the invention to modulate the activity of T cells. For example, a compound of interest can be assayed for its ability to stimulate T cell activation or inhibit tumor growth. For example, a compound of the invention can be administered to a mammal such as a mouse, before or after challenge with a tumorigenic dose of transformed cells and the presence or size of growing tumors may be monitored. Alternatively, the frequency of antigen-specific T cells among splenocytes or peripheral blood lymphocytes obtained from the mammal at various times following administration of a compound of the invention can be monitored by staining with a labeled MHC:peptide tetramer constituted with the specific peptide of interest. See Altman J D et al. Science 274: 94-96 (1996).

The present invention also includes pharmaceutical compositions comprising a compound described above in combination with a suitable pharmaceutical carrier. Such compositions comprise a therapeutically effective amount of the compound and a pharmaceutically acceptable carrier or excipient. Such a carrier includes but is not limited to saline, buffered saline, dextrose, water, glycerol, ethanol, and combinations thereof. The formulation should suit the mode of administration.

The present invention also includes a method of modulating, i.e., either stimulating or inhibiting an immune response, comprising administering to an animal an effective amount of a compound or composition of the invention.

The compounds of the present invention may be administered in pharmaceutical compositions in combination with one or more pharmaceutically acceptable excipients. It will be understood that, when administered to a human patient, the total daily usage of the pharmaceutical compositions of the present invention will be decided by the attending physician within the scope of sound medical judgment. The specific therapeutically effective dose level for any particular patient will depend upon a variety of factors including the type and degree of the response to be achieved; the specific composition of another agent, if any, employed; the age, body weight, general health, sex and diet of the patient; the time of administration, route of administration, and rate of excretion of the composition; the duration of the treatment; drugs (such as a chemotherapeutic agent) used in combination or coincidental with the specific composition; and

like factors well known in the medical arts. Suitable formulations, known in the art, can be found in Remington's Pharmaceutical Sciences (latest edition), Mack Publishing Company, Easton, Pa.

The compound to be used in the therapy will be formulated and dosed in a fashion consistent with good medical practice, taking into account the clinical condition of the individual patient (especially the side effects of treatment with the compounds alone), the site of delivery of the compound, the method of administration, the scheduling of administration, and other factors known to practitioners. The “effective amount” of the compounds of the invention for purposes herein is thus determined by such considerations.

Pharmaceutical compositions of the invention may be administered orally, intravenously, rectally, parenterally, intracistemally, intradermally, intravaginally, intraperitoneally, topically (as by powders, ointments, gels, creams, drops or transdermal patch), bucally, or as an oral or nasal spray. The term “parenteral” as used herein refers to modes of administration which include intravenous, intramuscular, intraperitoneal, intrastemal, subcutaneous and intraarticular injection and infusion.

The pharmaceutical compositions are administered in an amount which is effective for treating and/or prophylaxis of the specific indication. In most cases, the dosage is from about 1 μg/kg to about 30 mg/kg body weight daily, taking into account the routes of administration, symptoms, etc. However, the dosage can be as low as 0.001 μg/kg.

As a general proposition, the total pharmaceutically effective amount of the compositions administered parenterally per dose will be in the range of about 1 μg/kg/day to 100 mg/kg/day of patient body weight, although, as noted above, this will be subject to therapeutic discretion. If given continuously, the composition is typically administered at a dose rate of about 1 μg/kg/hour to about 5 mg/kg/hour, either by 1-4 injections per day or by continuous subcutaneous infusions, for example, using a mini-pump. An intravenous bag solution or bottle solution may also be employed.

The compounds of the invention may also be suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include liposomally entrapped compositions of the present invention. Liposomes are prepared by methods known per se: DE 3,218,121; Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compositions that are known to be deleterious to polypeptides.

Generally, the formulations are prepared by contacting the compounds of the invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Suitable formulations, known in the art, can be found in Remington's Pharmaceutical Sciences (latest edition), Mack Publishing Company, Easton, Pa.

The carrier suitably contains minor amounts of additives such as substances that enhance isotonicity and chemical stability. Such materials are non-toxic to recipients at the dosages and concentrations employed, and include buffers such as phosphate, citrate, suαinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The compositions are typically formulated in such vehicles at a concentration of about 0.01 μg/ml to 100 mg/ml, preferably 0.01 μg/ml to10 mg/ml, at a pH of about 3 to 8. It will be understood that the use of certain of the foregoing excipients, carriers, or stabilizers will result in the formation of salts.

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The compounds of the invention ordinarily will be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized composition using bacteriostatic Water-for-Injection.

Dosaging may also be arranged in a patient specific manner to provide a predetermined concentration of activity in the blood, as determined by an RIA technique, for instance. Thus patient dosaging may be adjusted to achieve regular on-going trough blood levels, as measured by RIA, on the order of from 50 to 1000 ng/ml, preferably 150 to 500 ng/ml.

The compounds of the invention are useful for administration to any animal, preferably a mammal (such as apes, cows, horses, pigs, boars, sheep, rodents, goats, dogs, cats, chickens, monkeys, rabbits, ferrets, whales, and dolphins), and more preferably a human.

The invention also provides a pharmaceutical pack or kit comprising one or more containers filled with one or more of the ingredients of the pharmaceutical compositions of the invention. Associated with such containers can be a notice in the form prescribed by a governmental agency regulating the manufacture, use or sale of pharmaceuticals or biological products, which notice reflects approval by the agency of manufacture, use or sale for human administration. In addition, the compositions of the present invention may be employed in conjunction with other therapeutic compositions.

Other therapeutic compositions useful for administration along with a compound of the present invention include cytotoxic drugs, particularly those which are used for cancer therapy. Such drugs include, in general, alkylating agents, anti-proliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. As noted previously, one skilled in the art may make chemical modifications to the desired compound in order to make reactions of that compound more convenient for purposes of preparing conjugates of the invention.

The compounds of the invention can be used to treat tumor-bearing animals, including humans, to generate an immune response against tumor cells. The generation of an adequate and appropriate immune response leads to tumor regression in vivo. Such “vaccines” can be used either alone or in combination with other therapeutic regimens, including but not limited to chemotherapy, radiation therapy, surgery, bone marrow transplantation, etc. for the treatment of tumors. For example, surgical or radiation techniques could be used to debulk the tumor mass, after which, the vaccine formulations of the invention can be administered to ensure the regression and prevent the progression of remaining tumor masses or micrometastases in the body. Alternatively, administration of the “vaccine” can precede such surgical, radiation or chemotherapeutic treatment.

Alternatively, the compounds of the invention can be used to immunize or “vaccinate” tumor-free subjects to prevent tumor formation. With the advent of genetic testing, it is now possible to predict a subject's predisposition for certain cancers. Such subjects, therefore, may be immunized using a compound comprising one or more antigenic peptides derived from tumors.

Suitable preparations of such vaccines include injectables, either as liquid solutions or suspensions; solid forms suitable for solution in, or suspension in liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1 ′-2′-dipalmitoyl-sn-glycero-3-hydroxyphosphoryloxy)-ethylamine, GM-CSF, QS-21 (investigational drug, Progenics Pharmaceuticals,Inc.), DETOX (investigational drug, Ribi Pharmaceuticals), BCG, and CpG rich oligonucleotides.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

In an alternate embodiment, compounds of the present invention may be used in adoptive immunotherapeutic methods for the activation of T lymphocytes that are histocompatible with the patient. (for methods of adoptive immunotherapy, see, e.g., Rosenberg, U.S. Pat. No. 4,690,915, issued Sep. 1, 1987; Zarling, et al., U.S. Pat. No. 5,081,029, issued Jan. 14, 1992). Such T lymphocytes may be isolated from the patient or a histocompatible donor. The T lymphocytes are activated in vitro by exposure to the compound of the invention. Activated T lymphocytes are expanded and inoculated into the patient in order to transfer T cell immunity directed against the particular antigenic peptide or peptides.

The compounds of the present invention may be administered along with other compounds which modulate an immune response, for example, cytokines. The term “cytokine” refers to polypeptides, including, but not limited to, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, and IL-18), α interferons (e.g., IFNα), ω interferon (IFNω), βinterferons (e.g., IFNβ), γ interferons (e.g., IFNγ), τ interferon (IFNτ), colony stimulating factors (CSFs, e.g., CSF-1, CSF-2, and CSF-3), granulocyte- macrophage colony stimulating factor (GMCSF), transforming growth factor (TGF, e.g.,.TGFα and TGFβ), and insulin-like growth factors (IGFs, e.g., IGF-I and IGF-II).

The compounds of the invention may also be employed in accordance with the present invention by expression of such compounds, especially MHC-antibody fusion compounds, in vivo, which is often referred to as “gene therapy.”

Polynucleotide that encodes a compound of this invention that is a direct fusion of antibody and an MHC class I α3 domain, as well as a polynucleotide encoding a β₂-microglobulin fusion, may be introduced directly into cells by transfection or infection with a suitable vector so as to give rise to synthesis and secretion of that compound by the successfully transfected or infected cells. This can be accomplished by cotransfection with separate DNA vector constructs or by co-expression in the same vector. In a preferred embodiment two constructs are prepared, an immunoglobulin-MHC class I α3 chain fusion and a specific peptide-β₂-microglobulin fusion.

Thus, for example, cells from a patient may be engineered with a polynucleotide (DNA or RNA) encoding a compound of the invention ex vivo, with the engineered cells then being provided to a patient to be treated with the compounds. Such methods are well-known in the art. For example, cells may be engineered by procedures known in the art by use of a retroviral particle containing RNA encoding a compound of the present invention.

Similarly, cells may be engineered in vivo for expression of a compound in vivo by, for example, procedures known in the art. As known in the art, a producer cell for producing a retroviral particle containing RNA encoding the compound of the present invention may be administered to a patient for engineering cells in vivo and expression of the polypeptide in vivo. These and other methods for administering a polypeptide of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle. Examples of other delivery vehicles include an HSV-based vector system, adeno-associated virus vectors, pox viruses, and inert vehicles, for example, dextran coated ferrite particles.

Retroviruses from which the retroviral plasmid vectors hereinabove mentioned may be derived include, but are not limited to, lentiviruses, Moloney Murine Leukemia virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The nucleic acid sequence encoding the compound of the present invention is under the control of a suitable promoter. Suitable promoters which may be employed include, but are not limited to, adenoviral promoters, such as the adenoviral major late promoter; or heterologous promoters, such as cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters.

The retroviral plasmid vector is employed to transduce packaging cell lines to form producer cell lines. Examples of packaging cell lines which may be transfected include, but are not limited to, the PE501, PA317, 2-2, 2-AM, PA12, T19-14x, VT-19-17-H2, 2CRE, 2CRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In certain embodiments, the polynucleotide constructs may be delivered as naked polynucleotides. By “naked” polynucleotides is meant that the polynucleotides are free from any delivery vehicle that acts to assist, promote, or facilitate entry into the cell, including viral sequences, viral particles, liposome formulation, lipofectin, precipitating agents and the like. Such methods are well known in the art and described, for example, in U.S. Pat. Nos. 5,593,972, 5,589,466, and 5,580,859.

The naked polynucleotides used in the invention can be those which do not integrate into the genome of the host cell. These may be non-replicating sequences, or specific replicating sequences genetically engineered to lack the genome-integration ability. Alternatively, the naked polynucleotides used in the invention may integrate into the genome of the host cell by, for example, homologous recombination, as discussed below. Preferably, the naked polynucleotide construct is contained in a plasmid. Suitable expression vectors for delivery include, but are not limited to, vectors such as pRSVcat (ATCC 37152), pSVL and MSG (Pharmacia, Uppsala, Sweden), pSV2dhfr (ATCC 37146) and pBC12MI (ATCC 67109). Additional suitable plasmids are discussed in more detail above.

The naked polynucleotides can be administered to any tissue (such as muscle tissue) or organ, as described above. In another embodiment, the naked polynucleotides are administered to the tissue surrounding the tissue of origin. In another embodiment, the naked polynucleotides are administered systemically, through intravenous injection.

For naked polynucleotide injection, an effective dosage amount of polynucleotide will be in the range of from about 0.05 μg/kg body weight to about 50 mg/kg body weight. Preferably, the dosage will be from about 0.005 mg/kg to about 20 mg/kg and more preferably from about 0.05 mg/kg to about 5 mg/kg. The appropriate and effective dosage of the polynucleotide construct can readily be determined by those of ordinary skill in the art and may depend on the condition being treated and the route of administration.

The constructs may also be delivered with delivery vehicles such as viral sequences, viral particles, liposome formulations, lipofectin, precipitating agents, etc. Such methods of delivery are known in the art. For example, the polynucleotide construct can be delivered specifically to hepatocytes through the method of Wu et al., J. Biol. Chem. 264:6985-16987 (1989).

In certain embodiments, the polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416); MRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA (1987) 84:7413-7416). Other commercially available liposomes include transfectace (DDAB/DOPE) and DOTAP/DOPE (Boehringer).

Other cationic liposomes can be prepared from readily available materials using techniques well known in the art. See, e.g. PCT Application No. WO 90/11092 for a description of the synthesis of DOTAP (1,2-bis (oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphosphatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is ciculated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well knowvn in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophiys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166).

Additional examples of useful cationic lipids include dipalmitoyl-phophatidylethanolamine 5-carboxyspen-nylamide (DPPES); 5-carboxyspermylglycine dioctadecylamide (DOGS); dimethyldioctdecyl-ammonium bromide (DDAB); and (+)-N,N-dimethyl-N-[2-(sperminecarboxamido) ethyl]-2,3-bis(dioleyloxy)-1-propaniminium pentahydrochloride (DOSPA). Non-diether cationic lipids, such as DL-1,2-dioleoyl-3-dimethylaminopropyl-β-hydroxyethylammonium (DORI diester), 1,2-O-dioleyl-3-dimethylaminopropyl-β-hydroxyethylammonium (DORIE diether), 1-O-oleyl-2-oleoyl-3-dimethylaminopropyl-β-hydroxyethylammonium (DORI ester/ether), and their salts promote in vivo gene delivery. Cationic cholesterol derivatives such as, {3β[N-N,N′-dimethylamino)ethane]-carbomoyl}-cholesterol (DC-Chol), are also useful.

Preferred cationic lipids include: (±)-N-(2-hydroxyethyl)-N,N-dimethyl-2, 3-bis(tetradecyloxy)-1-propaniminium bromide; 3,5-(N,N-di-lysyl) diamino-benzoylglycyl-3-(DL-1,2-dioleoyl-dimethylaminopropyl-β-hydroxyethylamine) (DLYS-DABA-GLY-DORI diester); 3,5-(NN-dilysyl)-diaminobenzoyl-3-(DL-1,2-dioleoyl-dimethylaminopropyl-β-hydroxyethylamine) (DLYS-DABA-DORI diester); and 1,2-dioleoyl-sn-glycero-3-phosphoethanolamine. Also preferred is the combinations of the following lipids: (±)-N-(2-hydroxyethyl)-N,N-dimethyl-2,3-bis (tetradecyloxy)-1-propaniminium bromide and 1,2-dioleoyl-sn-glycero-3-phosphoethanolamine; and (+)-N-(2-hydroxyethyl)-N,N-dimethyl-2,3-bis(tetradecyloxy)-1-propaniminium bromide, and 1,2-dioleoyl-sn-glycero-3-phosphoethanolamine in a 1:1 ratio.

The lipid formulations may have a cationic lipid alone, or also include a neutral lipid such as cardiolipin, phosphatidylcholine, phosphatidylethanolamine, dioleoylphosphatylcholine, dioleoylphosphatidyl-ethanolamine, 1,2-dioleoyl-sn-glycero-3-phosphatidylethanolamine (DOPE), sphingomyelin, and mono-, di- or tri-acylglycerol).

Lipid formulations may also have cationic lipid together with a lysophosphatide. The lysophosphatide may have a neutral or a negative head group. Useful lysophosphatides include lysophosphatidylcholine, lysophosphatidyl-ethanolamine, and 1-oleoyl lysophosphatidylcholine. Lysophosphatide lipids are present Other additives, such as cholesterol, fatty acid, ganglioside, glycolipid, neobee, niosome, prostaglandin, sphingolipid, and any other natural or synthetic amphiphiles, can be used. A preferred molar ratio of cationic lipid to neutral lipid in these lipid formulations is from about 9:1 to about 1:9; an equimolar ratio is more preferred in the lipid-containing formulation in a 1:2 ratio of lysolipid to cationic lipid.

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 provide cationic lipids for use in transfecting DNA into cells and mammals. U.S. Pat. Nos. 5,589,466, 5,693,622, 5,580,859, 5,703,055, and international publication no. WO 94/9469 provide methods for delivering DNA-cationic lipid complexes to mammals.

In certain other embodiments, cells are engineered, ex vivo or inl vivo, with the polynucleotide operably linked to a promoter contained in an adenovirus vector. Adenovirus can be manipulated such that it encodes and expresses the desired gene product, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the El region of adenovirus and constitutively express Ela and Elb, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the polynucleotide of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells.

Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host cell integration. The polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells, either ex vivo or in vivo. The transduced cells will contain the polynucleotide construct integrated into its genome, and will express the molecule of interest.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of the liver. Administration of a composition locally within the area of the liver refers to injecting the composition centimeters and preferably, millimeters within the liver.

Another method of local administration is to contact a polynucleotide-promoter construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site, for example, ligands for targeting the vehicle to a tissue of interest. Targeting vehicles for other tissues and organs are well known to skilled artisans.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Direct administration of a DNA construct coding for a compound of the invention can be suitably accomplished for expression of the fusion compound within cells of the subject. Also, rather than directly administering nucleic acids coding for a compound of the invention to a subject, host compatible cells into which such nucleic acids have been introduced may be administered to the subject. Upon administration to a subject, such engineered cells can then express in vivo the compound of the invention. Such engineered cells can be administered to a subject to induce an immune response or alternatively to suppress an immune response, as disclosed herein.

A treatment method for suppression of an immune response provides for administration of a compound of the invention in which the peptide is a TCR antagonist or partial agonist. See Sette et al., Ann. Rev. Immunol. 12:413-431 (1994)). Peptides that are TCR antagonists or partial agonists can be readily identified and selected by the in vitro protocols identified above. A compound of the invention that contains a peptide that is a TCR antagonist or partial agonist is particularly preferred for treatment of allergies and autoimmune diseases.

Immunosuppressive therapies of the invention also may be used in combination with other known immunosuppressive agents such as anti-inflammatory drugs to provide a more effective treatment of a T cell-mediated disorder. For example, other immunosuppressive agents useful in conjunction with the compounds of the invention include anti-inflammatory agents such as corticosteroids and nonsteroidal drugs.

The invention also provides methods for invoking an immune response in a mammal such as a human, including vaccinating a mammal with a compound or composition described herein.

The compounds of the invention are useful for raising an immune response and treating hyperproliferative disorders. Examples of hyperproliferative disorders that can be treated by the compounds of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated by the compounds of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The compounds of the present invention are also useful for raising an immune response against infectious agents. Viruses are one example of an infectious agent that can cause disease or symptoms that can be treated by the compounds of the invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia.

Similarly, bacterial or fungal agents that can cause disease or symptoms and that can be treated by the compounds of the invention include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lymne Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections.

Moreover, parasitic agents causing disease or symptoms that can be treated by the compounds of the invention include, but are not limited to, the following families: amebiasis, babesiosis, coccidiosis, cryptosporidiosis, dientamoebiasis, dourine, ectoparasitic, giardiasis, helminthiasis, leishmaniasis, theileriasis, toxoplasmosis, trypanosomiasis, and trichomonas.

Additionally, the compounds of the invention are useful for treating autoimmune diseases. An autoimmune disease is characterized by the attack by the immune system on the tissues of the victim. In autoimmune diseases, the recognition of tissues as “self”apparently does not occur, and the tissue of the afflicted subject is treated as an invader—i.e., the immune system sets about destroying this presumed foreign target. The compounds of the present invention are therefor useful for treating autoimmune diseases by desensitizing the immune system to these self antigens by provided a TCR signal to T cells without a costimulatory signal or with an inhibitory signal.

Examples of autoimmune diseases which may be treated using the compounds of the present invention include, but are not limited to Addison's Disease, hemolytic anemia, antiphospholipid syndrome, rheumatoid arthritis, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, polyendocrinopathies, purpura, Reiter's Disease, Stiff-Man Syndrome, autoimmune thyroiditis, systemic lupus erythematosus, autoimmune pulmonary inflammation, Guillain-Barre Syndrome, insulin dependent diabetes mellitis, autoimmune inflammatory eye disease, autoimmune hemolysis, psoriasis, juvenile diabetes, primary idiopathic myxedema, autoimmune asthma, scleroderma, chronic hepatitis, hypogonadism, pernicious anemia, vitiligo, alopecia areata, Coeliac disease, autoimmune enteropathy syndrome, idiopathic thrombocytic purpura, acquired splenic atrophy, idiopathic diabetes insipidus, infertility due to antispermatazoan antibodies, sudden hearing loss, sensoneural hearing loss, polymyositis, autoimmune demyelinating diseases, traverse myelitis, ataxic sclerosis, progressive systemic sclerosis, dermatomyositis, polyarteritis nodosa, idiopathic facial paralysis, cryoglobulinemia, inflammatory bowel diseases, Hashimoto's disease, adrenalitis, hypoparathyroidism, and ulcerative colitis.

Similarly, allergic reactions and conditions, such as asthma (particularly allergic asthma) or other respiratory problems, may also be treated by compounds of the invention. Moreover, the compounds of the invention can be used to treat anaphylaxis, hypersensitivity to an antigenic molecule, or blood group incompatibility.

The compounds of the invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues.

The administration of the compounds of the invention that inhibit an immune response may be an effective therapy in preventing organ rejection or GVHD.

The compounds of the invention which can inhibit an immune response are also usefuil for treating and/or preventing atherosclerosis; olitis; regional enteritis; adult respiratory distress syndrome; local manifestations of drug reactions, such as dermatitis, etc.; inflammation-associated or allergic reaction patterns of the skin; atopic dermatitis and infantile eczema; contact dermatitis; psoriasis; lichen planus; allergic enteropathies; allergic rhinitis; bronchial asthma; hypersensitivity or destructive responses to infectious agents; poststreptococcal diseases, e.g. cardiac manifestations of rheumatic fever, and the like.

Further, the compounds of the invention can be used as a male or female contraceptive. For example, a compound of the invention which is useful as a male contraceptive comprises as the antigenic peptide a peptide derived from PH30 beta chain sperm surface protein. See U.S. Pat. No. 5,935,578. A compound of the invention which is useful as a female contraceptive may comprise as the antigenic peptide a peptide derived from the human ZP2 or the human ZP3 protein. See U.S. Pat. No. 5,916,768.

Having generally described the invention, the same will be more readily understood by reference to the following examples, which are provided by way of illustration and are not intended as limiting.

### EXAMPLES

### Example 1

### Construction of Human IgG3-Avidin Fusion Antibodies

The construction of a human IgG3-Avidin fusion antibody with specificity for the hapten. dansyl has been previously described (S. U. Shin et al., J. Imnmunology 158: 4797-4804 (1997)). The objective of this work is to replace the portion of this molecule that provides binding specificity for dansyl (V_{H} and V_{L} domains) with the homologous domains (V_{H} and V_{L}) from antibodies that are specific for cell surface molecules. These resulting antibodies will retain the ability to bind biotinylated tetrameric MHC complexes, and will allow for the targeting of these tetramers to the cell type of interest (for example, professional antigen-presenting cells, T cells, tumor cells, epithelial cells, or fibroblasts).

Monoclonal antibodies specific for DC specific molecules such as CD83, CMRF-44, CMRF-56, DEC205, and BDCA-2, BDCA-3, BDCA-4,; for T cell specific molecules such as CD28, CTLA-4, and CD25; and for tumor specific molecules such as Muc1, and Her2/neu have been isolated. To construct compounds that will target MHC class I α3 complexes to cells expressing these molecules, the genes that encode the V_{H} and V_{L} domains of antibodies specific for these molecules are isolated from the hybridoma cells that produce the specific antibodies. The heavy and light chain variable regions of the anti-dansyl avidin antibody are then replaced with these variable region genes.

Hybridoma cells secreting antibodies specific for the cell markers of interest are used as the source of the variable region genes. Messenger RNA is isolated from these hybridomas, converted into double stranded cDNA, ligated into a plasmid vector, and transformed into bacteria in order to generate a cDNA library. This cDNA library is screened using a probe derived from the Constant (C) region of the Ig Heavy chain, and separately with a probe derived from the C region of the Ig light chain, using the ClonCapture cDNA Selection System (Clontech, Palo Alto, Calif.). Clones recombinant for the Ig cDNA are sequenced in order to determine the sequence of the heavy and light chain variable region genes. Once these full-length cDNAs (containing the coding region for the entire Ig) have been isolated, the next step is to replace the variable region genes of the anti-dansyl antibody with these newly isolated variable region genes.

The cDNA containing the heavy chain LVDJ domain of the antibody is modified by PCR to include an NheI site at the 5′ end, and an intron splice donor (SD) sequence (GTAAGT) and XbaI site at its 3′ end. The sequence of the sense primer is 5′ AAT GCT AGC N₍₁₂₋₂₀₎ (SEQ ID NO:36) and the antisense primer is 5′ ATT TCT AGA ACT TAC N₍₁₂₋₂₀₎ (SEQ ID NO:37). The unknown nucleotides (N) in the primers are designed according to the sequence of the Leader sequence (including the ATG start codon) (sense primer), or to the Joining Segment (J) (antisense primer). Following digestion with NheI and XbaI, this LVDJ SD PCR product is inserted into the NheI and XbaI sites of expression vector pcDNA3.1/Hygro(−)(Invitrogen), creating pcDNA3.1/Hygro/IgVH. The gene encoding the heavy chain of the anti-Dansyl IgG3-Avidin antibody is contained in plasmids pAG3520, pAG3513, and pAG3517. pAG3520 contains CH1-H-CH2-CH3; pAG3513 contains CH1-H; and pAG3517 contains CH1. The Ig-avidin portion of this molecule is excised from each vector by digestion with SalI and BamHI. This IgG3-avidin cassette is inserted into the XhoI/BamHI sites of pcDNA3.1/Hygro/IgVH (SalI and XhoI leave complementary overhangs), creating pcDNA3.1/IgVH/X, wherein X indicates CH1-Avidin, H-Avidin, or CH3-Avidin. Following transcription, the splice donor sequence at the 3′ end of the LVDJ is spliced in frame with the splice acceptor sequence at the 5′ end of the CH1 exon. The spliced mRNA encodes an human IgG3-avidin fusion protein.

The cDNA containing the light chain LVJ domain of the antibody is modified by PCR to include an NheI site at the 5′ end, and an intron splice donor (SD) sequence (GTAAGT) and XbaI site at its 3′ end. The sequence of the sense primer is 5′ AAT GCT AGC N₁₂₋₂₀₎ (SEQ ID NO:35) and the antisense primer is 5′ ATT TCT AGA ACT TAC N₍₁₂₋₂₀₎ (SEQ ID NO:36). The unknown nucleotides (N) in the primers are designed according to the sequence of the leader sequence (including the ATG start codon) (sense primer), or to the Joining Segment (J) (antisense primer). Following digestion with NheI and XbaI, this LVJ SD PCR product is inserted into the NheI and XbaI sites of expression vector pcDNA3.1/Neo(−)(Invitrogen), creating pcDNA3.1/Neo/IgVL. The gene encoding the constant (C) Domain of the human Kappa Ig is available in vector pCN101. The coding region of the human IgK C gene is isolated from this vector by PCR with a sense primer containing an XbaI site (5′ AATTCTAGAGTCTGTCCCTAACATGCCC (SEQ ID NO:37)), and a KpnI site on the antisense primer (5′ AAAGGTACCT GGAACTGAGGAGCAGGTG (SEQ ID NO:38)).

Following digestion with XbaI and KpnI, the IgCκ coding region is inserted into the XbaI and KpnI sites of pcDNA3.1/neo/IgVL, resulting in pcDNA3.1/neo/IgVL/K. Following transcription, the splice donor sequence at the 3′ end of the LVJ is spliced in frame with the splice acceptor sequence at the 5′ end of the K exon. The spliced nmRNA encodes a human kappa light chain variable region fuision protein of an antibody.

Transfection, for example, of both pcDNA3.1/IgVH/IgG3-Avidin and pcDNA3.1/neo/IgVL/K into a non-Ig secreting B cell line generates a hybridoma that secretes antibody-avidin fusion molecules that are specific for the desired molecule. Biotinylated MHC class I α3 can be assembled into polymeric complexes on these antibody-avidin fusion proteins in exactly the same way previously described for assembly of MHC:peptide tetramers on free streptavidin (Altman, J. et al., Science 274:94-96 (1996); Boniface, J. J. et al., Immunity 9:459-66 (1998)).

### EXAMPLE 2

### Chimeric Ig Heavy Chain Fusion To α3 of HLA-A*0201

In this example, a chimeric IgG1 heavy chain fragment capable of assembling with an immunoglobulin light chain to form a single antigen binding Fab fragment and containing one, two, or several copies (multimerized) of the α3 domain of HLA-A*0201 attached in-frame to the 3′ end of the Ig heavy chain fragment transcript is created.

In examples 2 and 3, plasmid DNA is used as the template for the PCR reactions. However, amplification from pooled cDNA using the primers described would likely also be possible to those skilled in the art.

The complete genes described in Examples 2 and 3 are designed for insertion into the expression vector pIRESbleo3 (Clontech). This strategy is not limited to the use of pIRESbleo3. Specifically, the use of other expression vectors simply requires re-engineering of the restriction digestion sites flanking the complete construct (NotI and BamHI in the case of the chimeric heavy chain α3-A*0201 and ClaI and BamHI in the case of the chimeric CMV-β2-microglobulin). These methods are well known to those practiced in the art.

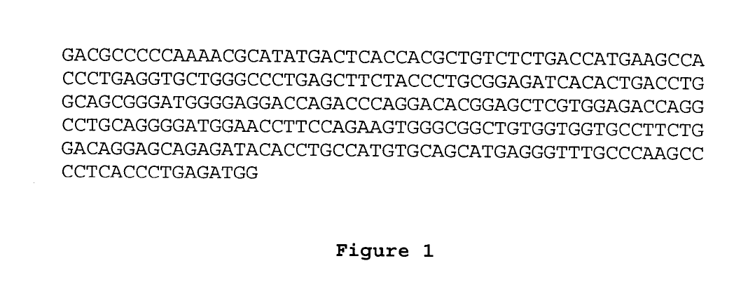

A. Production of a single copy of the α3 domain of HLA-A*0201. Plasmid DNA encoding HLA-A*0201 is obtained from Open Biosystems Inc. (Cat.#: MHS1011, OBS#: 169857, Source ID: 2821717, IMAGE ID: 2821717). The nucleotide sequence of the α3 domain of HLA-A*0201 is shown in FIG. 1 (SEQ ID NO:1).

The α3 domain is amplified using the following primers. The forward primer has a non-homologous region designed to allow overlap extension PCR with the 3′ end of the Ig sequence described below: 5′ GGAGGCGGTTCTTCAGACGCCCCCAAAACG. 3′ (SEQ ID NO:39). The reverse primer contains a stop codon and a linker containing a BamHI site: 5′ CGGGATCCCGTCACCATCTCAGGGTGAGG 3′ (SEQ ID NO:40). The primers described can be modified to include linker regions of various length and sequence between the 3′ end of the Ig fragment and the α3 domain by methods well known to those practiced in the art.

B. Production of an α3 multimer domain of HLA-A*0201. One. contiguous nucleotide construct is created which codes for two or more α3 domains of HLA-A*0201. The construct is prepared in such a way as to facilitate overlap extension PCR of the 5′ end of this construct with the 3′ end of the assembled Ig heavy chain construct described below. Insertion into the pIRESbleo (Clontech) expression construct is directed through a BamHI site at the 3′ end. The primers described can be modified to include linker regions of various length and sequence between the α3 domains by methods well known to those practiced in the art. This allows the physical distance between α3 domains to be optimized to increase the functional avidity of multimeric α3 domains for β2-microglobulin.

Creating a construct with exactly two repeats of the α3 domain. The following primers are used. The first forward primer (labeled primer 1) has a non-homologous region designed to allow overlap extension PCR with the 3′ end of the Ig sequence described below: 5′ GGAGGCGGTTCTTCAGACGCCCCCAAAACG 3′ (SEQ ID NO:39). The first reverse primer (labeled primer 2) contains a linker with an EcoRI site: Anti-sense 5′ GGAATTCTGAAGAACCGCCTCCCCA TCTCAGGGTGAGG 3′ (SEQ ID NO:41). The second forward primer (labeled primer 3) has a linker containing an EcoRI site: 5′ GGAATTCGGAGGCGGTTCTTCAGACGCCCCCAAAACG 3′ (SEQ ID NO:42). The second reverse primer contains a stop codon and has a linker containing a BamHI site: 5′ CGGGATCCCGTCACCATCTCAGGGTGAGG 3′ (SEQ ID NO:40).

Amplify using PCR from a plasmid containing HLA-A*0201 using primers 1 and 3 (referred to as OE-α3). PCR from a plasmid containing HLA-A*0201 using primers 4 and 2 (referred to as EB-α3). Gel purify the amplified products from each reaction. Digest the two products with EcoRI and ligate. Gel purify ligated product. The dimeric α3, OB-α3(2×), is then ready for overlap extension PCR with the Ig molecule described in section C below.

Creating a construct with n number of repeats of the α3 domain.

Step 1 (fragment A). PCR amplify the α3 domain using the following primers. The forward primer has a non-homologous region designed to allow overlap extension PCR with the 3′ end of the Ig sequence described below: 5′ GGAGGCGGTTCTTCAGACGCCCCCAAAACG 3′ (SEQ ID NO:39). The reverse primer contains a linker with an EcoRI site: 5′ GGAATTCTGAAGAACCGCCTCCCCATCTCAGGGTGAGG 3′ (SEQ ID NO:41). The fragment produced from this reaction is labeled OE-α3.

Step 2 (fragment B). PCR amplify the α3 domain using the following primers. The forward primer has a linker containing an EcoRi site: 5′ GGAATTCGGAGGCGGTTCTTCAGACGCCCCCAAAACG 3′ (SEQ ID NO:42). The reverse primer has a linker containing a BglII site: 5′ TCAGATCTTGAAGAACCGCCTCCCCATCTCAGGGTGAGG 3′ (SEQ ID NO:43). The fragment produced from this reaction is labeled EBg-α3.

The linker on the anti-sense primer can be any one of the following to accommodate ligation with the product of step 3: AscI, BglII, EcoRV, KpnI, NruI, XbaI. Additional α3 domains (fragment B1, B2, etc.) can be added by designing a new sense primer with a restriction digestion site corresponding to the anti-sense site from the prior B fragment and a new anti-sense primer with a different restriction digestion site selected from the group AscI, BglII, EcoRV, KpnI, NruI, or XbaI. These products would be ligated at the common site using standard protocols familiar to those practiced in the art. This step can be repeated as often as desired to extend the multimeric chain of α3 domains.

Step 3 (fragment C). The α3 domain is amplified by PCR using the following primers. The forward primer has a linker containing a restriction site that is the same as the restriction site employed in the fragment B reverse primer used to add the penultimate α3 domain. For this example, it is assumed that this is BglII: 5′ GAAGATCTGGAGGCGGTTCTTCAGACGCCCCCAAAACG 3′ (SEQ ID NO:44). The reverse primer has a linker containing a stop codon and a BamHI site: 5′ CGGGATCCCGTCACCATCTCAGGGTGAGG 3′ (SEQ ID NO:40).

The product from this reaction is labeled BgB-α3.

The BglII site on the sense primer can be changed to any one of AscI, BglII, EcoRV, KpnI, NruI, XbaI restriction sites in order to accommodate singular or multimerized products from step 2 (B fragment). This site will always match the site engineered into the last anti-sense primer from the B fragment.

Step 4. Gel purify products from all reactions. Ligate the OE-α3 to EBg-α3. Gel purify this ligated product and ligate with the gel purified BgB-α3. Gel purify ligated product.OB-α3(3×) is then ready for overlap extension PCR with the Ig molecule described in section C below.

The primers described for assembly of multimeric α3 domains can be modified to include linker regions of various length and sequence between each of the α3 domains by methods well known to those practiced in the art.

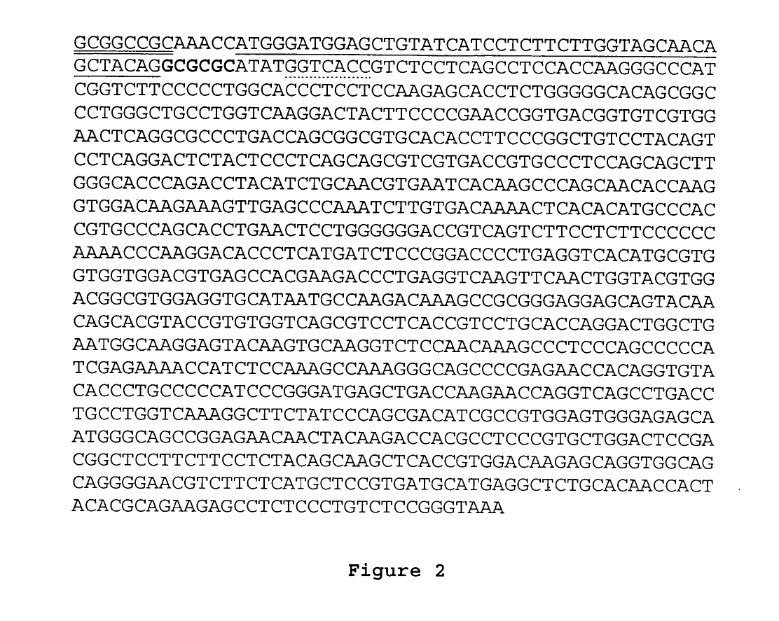

C. Assembled Ig Gamma Heavy Chain. One contiguous nucleotide construct is created which codes for a human Ig Gamma Heavy Chain V-CH1 fragment after insertion into an expression vector. The construct will be prepared to accept any immunoglobulin heavy chain variable gene sequence through a multiple cloning site situated between the signal sequence (SS) and constant region (CH1). The construct is prepared in such a way as to facilitate overlap extension PCR with the 5′ end of singular or multimerized α3 domain(s) of HLA-A*0201 and insertion into the pIRESbleo3 expression construct through a NotI site at the 5′ end. The nucleotide sequence of the assembled Ig Gamma Heavy Chain is shown in FIG. 2 (SEQ ID NO:2).

Human Ig Gamma1 Heavy Chain DNA is used as a template (Open Biosystems Inc.; Cat.#: MHS1011, OBS#: 61678, Source ID: 4308411, IMAGE ID: 4308411). PCR amplify using the following primers: 5′ AATTGCGGCCGCAAACCATGGGATGGAGCTGTATCATC 3′(SEQ ID NO:45) and 5′ TGAAGAACCGCCTCCTTTAC CCGGAGACAGGGA 3′ (SEQ ID NO:46).

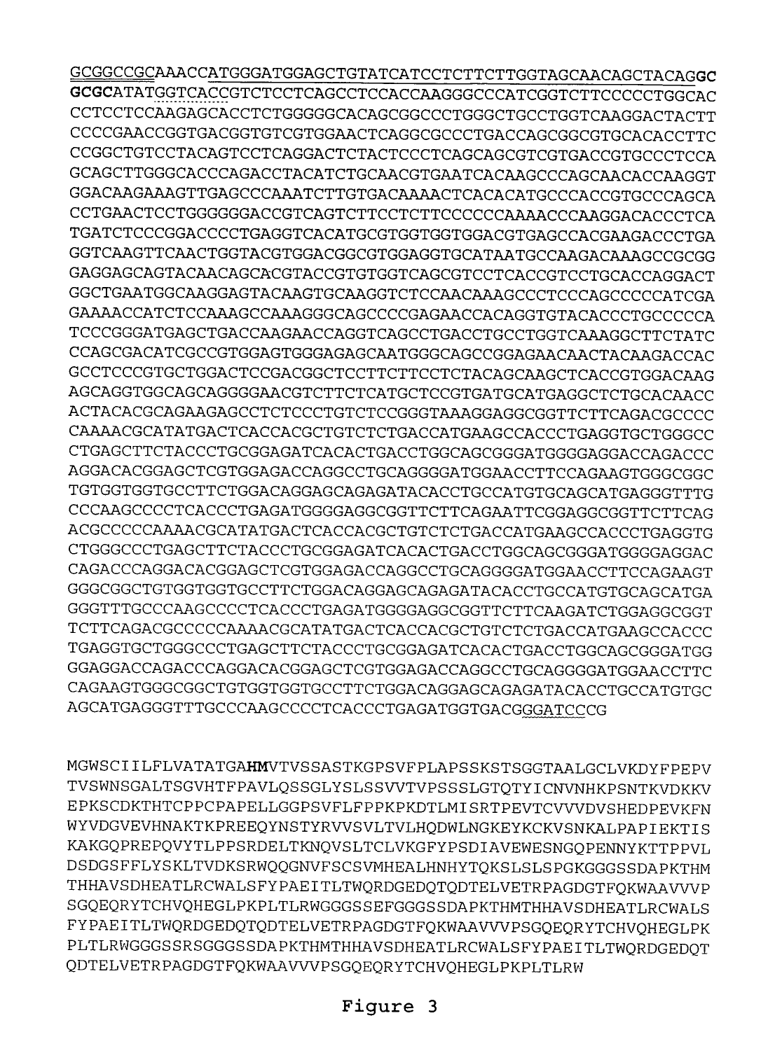

Completed and Assembled Chimeric protein, Ig-α3. The fully assembled chimeric immunoglobulin heavy chain fragment α3-A*0201 fusion is generated by overlap extension PCR using the products from section A combined with C or the product of B combined with C. Nucleotide and protein sequence is presented without an immunoglobulin heavy chain variable region V-gene in FIG. 3 (SEQ ID Nos: 3 and 4). Any given V-gene can be inserted between the BssHII (bold) and BstEII (dashed underline) sites.

### EXAMPLE 3

### Chimeric CMV Peptide-β2-Microglobulin

In this example, a chimeric CMV peptide-β32-microglobulin fusion protein is made which is capable of interacting with the α3 domain of HLA-A*0201 described above in Example 2. The chimeric protein will contain coding region for a class I restricted Human Cytomegalovirus (CMV) peptide epitope (CMV 495-503) and, for this example, includes the β2-microglobulin signal sequence to allow synthesis and assembly in eukaryotic cells. A related fragment without the signal sequence can be assembled for expression in bacteria employing methods well known to those practiced in the art.

A. Assembling the β₂-microglobulin signal sequence (“A” fragment). Oligonucleotides corresponding to the following sequences are synthesized: Sense 5 ′-CCATCGATATGTCTCGCTCCGTGGCCTT AGCTGTGCTCGCGCTACTCTCTCTTTCTGGCCTGGAGGCTAACCTGG TGCCCATG -3′ (SEQ ID NO:47) (containing a ClaI restriction site, a β2-microglobulin signal sequence and nucleotides 1-15 of the CMV epitope); Anti-sense 5′-CATGGGCACCAGGTTAGCCTCCAGGCC AGAAAGAGAGAGTAGCGCGAGCACAGCTAAGGCCACGGAGCGAG ACATATCGATGG-3′ (SEQ ID NO:48).

Double stranded β2-microglobulin signal sequence is generated by resuspending and mixing the custom oligonucleotides at equimolar concentration (100 mM). The mixture is then heated to 95° C. for 2 minutes and allowed to gradually cool to 25° C. over a period of 45 minutes. The double stranded complex is then gel purified on a 2% agarose gel. The 5′ end contains a ClaI endonuclease restriction site designed to allow for insertion into an expression construct. The 3′ end of the oligonucleotide is designed to allow for overlap extension PCR with the fragment described below.

B. Assembling the entire CMV peptide epitope, a 15 amino acid linker, and 15 nucleotides of the β₂-microglobulin gene (“B” fragment). Oligonucleotides corresponding to the following sequences are synthesized: Sense 5′-AACCTGGTGCCCATGGTGGCTACGGTTG GAGGTGGGGGAGGCGGATCAGGAGGCTCAGGTGGGTCAGGAGGCA TCCAGCGTACTCCA-3′ (SEQ ID NO:49) (including the complete CMV epitope, a 15 amino acid linker and a first 15 nucleotide fragment of the body of the β₂-microglobulin gene); Anti-sense 5′-TGGAGTACGCTGGATGCCTCCTGACCCACCTGAGCCTCCTGATCCG C CTCCCCCACCTCCAACCGTAGCCACCATGGGCACCAGGTT-3′ (SEQ ID NO:50).

Double stranded middle fragment is generated by resuspending and mixing these custom oligonucleotides at equimolar concentration (100mM). The mixture is then heated to 95° C. for 2 minutes and allowed to gradually cool to 25° C. over a period of 45 minutes. The double stranded complex is then gel purified on a 2% agarose gel. The 5′ end is designed to allow for overlap extension PCR with the fragment described in the section above. The 3′ end is designed to allow for overlap extension PCR with the fragment described in the section below.

C. Creating a DNA fragment comprising the body (minus the signal sequence) of the human β₂-microglobulin gene (“C” fragment). The fragment is generated by standard PCR using plasmid DNA as template (Source: Open Biosystems Inc.; Cat.#: EHS1001, OBS#: 26266, Source ID: 5502428, IMAGE ID: 5502428) and the following primers: Sense 5′-ATCCAGCGTACTCCAAAGATT-3′ (SEQ ID NO:51); Anti-sense 5′-CGGGATCCTTACATGTCTCGATCCCACTT-3′ (SEQ ID NO:52) (includes a BainHI restriction site). The PCR product is gel purified according to standard procedure.

D. Assembling the complete CMV peptide-β2-microglobulin Chimera. Step 1. The double stranded A and B fragments are assembled in an overlap extension PCR according to standard protocols well known to those practiced in the art. The resulting product is 155 nucleotides in length. This product is gel purified according to standard protocol.

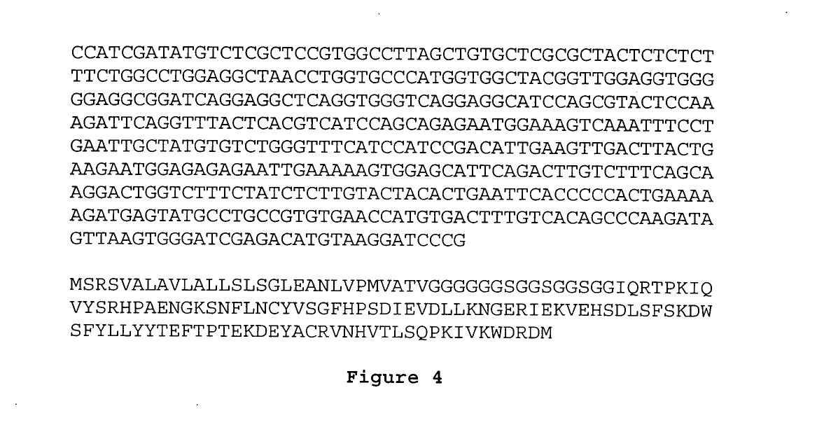

Step 2. The product from Step 1 is used in an overlap extension PCR reaction with the gel purified fragment C according to standard protocol. The resulting product is 440 nucleotides in length and is gel purified. This product is then ready for digestion with ClaI and BamHI and insertion into ClaI/BamHI sites of an expression construct such as pIRESbleo3 (Clontech). The complete sequence of the chimeric construct is shown in FIG. 4 (SEQ ID Nos: 5 and 6).

### EXAMPLE 4

### Assay for the In Vitro Activity of Compounds of the Invention Targeted to Dendritic Cells

Dendritic cells are the most potent stimulators of T cell responses identified to date. To test in vitro activity of compounds of the invention specifically targeted to dendritic cells, DC are incubated with the relevant compounds and assayed for the ability to activate human autologous T lymphocytes. Immature dendritic cells are prepared from healthy donors according to the method of Bhardwaj and colleagues (Reddy, A. et al., Blood 90:3640-3646 (1997)). Briefly, PBMC are incubated with neuraminidase-treated sheep erythrocytes and separated into rosetted T cell (ER+) and non-T cell (ER−) fractions. The ER+fraction is cryopreserved for later use. The ER-fraction (2×10⁶ cells per well) is cultured in serum-free RPMI medium containing 1000 U/ml rhGM-CSF, 1000 U/ml rhIL-4 and 1% autologous plasma. This medium is replenished every other day. At day 7, the, non-adherent immature DC are harvested from the culture and re-plated in maturation conditions (1000 U/ml GM-CSF, 1000 U/ml IL-4, 1% autologous plasma and 12.5-50% monocyte-conditioned medium) for 2-4 days. Cells manipulated in this manner have morphological and surface characteristics (CD83+) of mature DC.

Mature (or immature) DC are pulsed with compounds of the invention, or with free peptide or free MHC class I α3 complexes as controls for a short period followed by cocultivation with autologous T cells in 24 well plates for a period of 7-14 days. In some experiments, these may be total T lymphocytes, but it may also be desirable to fractionate CD8+T cells using magnetic separation systems (Miltenyi Biotech). Total T lymphocytes are incubated with the appropriate antibody-magnetic bead conjugates to isolate total CD8, naïve CD8+CD45RA+, or memory CD8+CD45RO+lymphocytes. For naive CD8 lymphocytes, a cytokine cocktail consisting of IL-2 (20 U/ml), IL-12 (20 U/ml), IL-18 (10 ng/ml), IFN-gamma (1 ng/ml) and a monoclonal antibody specific for IL-4 (50 ug/ml) is especially potent in enhancing DC activation of cytotoxic T cells in vitro. If T cells derive from a naive donor, then it is likely that two to four rounds of stimulation 10 to 14 days apart under these same conditions in vitro may be required for sufficient T cell expansion. Following the activation period, CTL activity is assessed in a 4 hour ⁵¹Cr release assay. Other in vitro assays of T cell activation include proliferation (measured by increases in ³H-Thymidine incorporation or colorimetric MTT assay), cytokine secretion (IFN-γ, TNF-α, GM-CSF, IL-2) measured by ELISA, ELISpot, or flow cytometric detection (Luminex bead system). Many of these methods are described in Current Protocols in Immunology (John Wiley & Sons, New York). These and other methods are well known to those practiced in the art. Enhancement of T cell responses to targeted compounds of the invention is determined by comparison to the response to equimolar concentrations of free peptide or untargeted MHC class I α3 complexes.

### EXAMPLE 5

### Assay for In Vivo T Cell Expansion Following Stimulation with Compounds of the Invention

The effect of targeted vaccine complexes on expansion of specific T cells in vivo in either humans or HLA transgenic mice is determined by recovering T cells before and at internals following immunization with a specific vaccine complex and determining the frequency of T cells specific for the vaccine complex by staining with tetrameric complexes of the same peptide:MHC. Tetramers comprising the same peptide MHC complex of interest are employed in a cell surface immunofluorescence assay as follows. HLA-transgenic mouse spleen, lymph node or peripheral blood cells (collected by tail or retro-orbital bleeding) or human PBMC (1-10×10⁵ cells per sample) are incubated on ice in the presence of azide with control or experimental tetramers for about 30 minutes. After washing 2-3 times with staining buffer (such as PBS 1% BSA, 0.1% azide) a secondary streptavidin-fluorochrome (FITC, PE, or other fluorochrome) conjugate is added. After incubating for about 30 minutes, the samples are again washed 2-3 times and immunofluorescence is detected using a flow cytometer. These data are compared to pre-vaccination flow cytometric profiles to determine percentage increase in T cell precursor frequency and are repeated multiple times during the course of an experiment or clinical trial.

### EXAMPLE 6

### In Vitro Assays for Tumoricidal Activity of T Cells Specifically Targeted to Tumors by Compounds of the Invention

To demonstrate the ability to redirect cytotoxic T cells to the desired tumor target, tumor cells are incubated with compounds of the invention comprised of a tumor-specific antibody linked to MHC class I α3 complexes which incorporate a peptide for which T cells are prevalent (for example, influenza matrix peptide 58-66). ⁵¹Cr (100 μci) is added during this 1 hour incubation to label the tumor cells. Following 2-3 washes, influenza specific CTL restricted to the appropriate MHC molecule are added at various effector to target (E:T) ratios in a 4 hr chromium release assay. Increased tumor lysis in the experimental sample containing compounds of the invention relative to control compounds with irrelevant MHC class I α3 complexes or non tumor-specific antibody linked to the relevant MHC class I α3 complexes demonstrates that the compound of interest successfully sensitizes tumors to lysis by CTL specific for influenza virus.

The experimental protocol described in the previous paragraph demonstrates redirection of cytotoxic effector function of influenza peptide-specific CTL to uninfected tumor cells by compounds of the invention that comprise a tumor specific antibody and influenza linked MHC class I α0.3 complexes. To demonstrate the ability of tumor cells treated with the same compound to activate influenza peptide-specific T cell response, total T cells from influenza immune individuals are stimulated in 24 well plates (1-2×10⁶ T cells per well ) with irradiated or mitomycin C treated tumor cells (1-10×10⁵) pulsed with compounds of the invention having influenza matrix peptide-β02-microglobulin bound in tumor-specific antibody targeted MHC class I α3 complexes. Cytokines such as IL-2, IL-7, IL-12, IL-18, IFN-γ may also be added to enhance activation of naïve CTL. Induction of cytotoxic T lymphocytes is assessed in a standard ⁵¹Cr release assay.

This same method of targeting MHC class I α3 complexes linked to viral peptides to the tumor cell surface can be employed to enhance MHC-restricted presentation of known tumor-specific peptides.

### EXAMPLE 7

### In vivo assays for tumoricidal activity of T cells specifically targeted to tumors by compounds of the invention

In a murine experimental model, compounds of the invention can be targeted to tumor cells through a naturally occurring or transfected tumor membrane marker. For example, BALB/c tumors such as EMT-6 (mammary carcinoma, Rockwell, S. C. et al., J. Natl. Cancer Inst. 49:735-749 (1972)), Line 1 (small cell lung carcinoma, Yuhas, J. M. et al., Cancer Res. 34:722-728 (1974)) or BCA (fibrosarcoma, Sahasrabudhe, D. M. et al., J. Immunology 151:6302-6310 (1993)) may be transfected with a model antigen (e.g. carcinoma embryonic antigen, CEA) for which antibodies are commercially available or easily made by the skilled artisan. Antibodies or antibody fragments specific for this model antigen may be linked to MHC class I α3 complexes associated with peptides that are either naturally occurring in that tumor, such as the L3 ribosomal protein peptide 48-56 expressed in association with H-2K^{d} in the BCA tumors, or a well-characterized pathogenic peptide known to induce a high frequency of high avidity T cells, such as the the HIV gp160IIIB peptide RGPGRAFVTI (SEQ ID NO:53) (Shirai, M. et al., J. Immunol. 148:1657 (1992)).

BALB/c (H-2^{d}) mice with established tumors and/or distant metastases expressing the targeted molecule (e.g. CEA) and that have been immunized with a vaccinia recombinant of HIV gp160IIIB are injected with gp160IIIB peptide-β2-microglobulin associated with MHC class I α3 domains linked to an anti-CEA antibody specificity for targeting to tumor cells. The effect on tumor growth of treatment with these compounds of the invention is monitored by caliper measurements every other day.

This analysis can be extended to human tumors implanted in immunodeficient (SCID, Rag-1^{-/-}, or Rag-1^{-/-} common γ chain double knockout) mice. Following establishment of tumors in vivo, mice receive an injection(s) of compounds of the invention specific for human tumor antigens conjugated to MHC class I α3 complexes with linked HLA-A2 restricted influenza peptide (or a control peptide). Influenza specific human CTL are adoptively transferred and tumor regression is monitored.

In clinical trials, a standard influenza vaccination may be added to the protocol to increase influenza specific CTL directed at the tumor by compounds of the invention comprising influenza peptide:MHC class I α3 complexes.

### EXAMPLE 8

### Effects of Compounds of the Invention on Antigen Stimulated T Cell Proliferation

In this example, T cells are obtained from immune BALB/c mice and the presence of specific T cells is revealed by restimulation in vitro with compounds of the invention. Mice are immunized with ovalbumin peptide (2 mg/ml in PBS) mixed with 600 jg CpG oligonucleotide, Carson, D. A. and Raz, E. J. Exp. Med. 186:1621-2 (1997) and incomplete Freund's adjuvant in a 1:1 v/v ratio. Fifty μl are injected s.c. into each side of the base of the tail.

Seven days after the last injection, lymph nodes (inguinal, paraaortic, cervical, axillary, brachial) are removed and homogenized to obtain a single cell suspension. Lymph nodes from individual mice within a group are processed separately. T cells are purified from lymph node populations by passage of cell suspensions over G-10 and nylon wool to remove accessory cells, and the resulting purified T cell populations incubated with Click's medium.

Activated B cells from BALB/c mice are used as antigen presenting cells in the proliferation assay. B cells are prepared by culturing spleen cells with 50 μg/ml of LPS for 48 to 72 hours at which time activated cells will be isolated by density gradient centrifugation on Lymphoprep. Activated B cells are then cultured with the compound of interest linked to a B cell targeting antibody for 3 hours, washed extensively, fixed with paraformaldehyde to inhibit proliferation of B cells, and added to purified T cells.

The proliferation assay is carried out in 96 well round bottom microtiter plates at 37° C., 5% CO₂ for 3-5 days. Wells are pulsed with 1 αCi of ³H-thymidine for 18 hours prior to termination of cultures and harvested using a Skatron cell harvester. Incorporation of ³H-thymidine into DNA as a measure of T cell proliferation are determined using an LKB liquid scintillation spectrometer. An increase in T cell proliferation following contact with B cells treated with the compound of the invention as compared to a negative control, indicates the compound of interest can stimulate immune responses in a peptide-specific manner.

Alternatively, IL-2 levels can be measured, as described above, at 24 and 48 hours.

### EXAMPLE 9

### Assay for Immune Induction or Suppression by MHC Fusion Complex

This example uses an animal model of immunization with ovalbumin peptide 323-339 and manipulation of the response to the peptide. The methodology of this example can be applied to a wide variety of compounds of the invention that contain a peptide which can modulate (i.e., suppress or induce) an immune response in an animal.

BALB/c mice (3 per group) are injected i.v. or i.p. with 100 μl of the compound of interest which contains OVA 323-339 as the antigenic peptide. At various times following introduction of the compund of interest, these mice are immunized according to the protocol of Example 10. Ovalbumin peptide (2 mg/ml in PBS) is mixed with 600 μg CpG oligonucleotide, Carson, D. 2587 A. and Raz, E. J. Exp. Med. 186:1621-2 (1997) and incomplete Freund's adjuvant in a 1:1 v/v ratio. Fifty μl are injected s.c. into each side of the base of the tail. Seven days after the last injection, lymph nodes (inguinal, paraaortic, cervical, axillary, brachial) are removed and homogenized to obtain a single cell suspension. Lymph nodes from individual mice within a group are processed separately. T cells are purified from lymph node populations by passage of cell suspensions over G-10 and nylon wool to remove accessory cells.

Antigen presenting cells are prepared from the spleens of naive BALB/c mice by homogenizing spleens to obtain a single cell suspension, lysis of erythrocytes using Gey's solution, treatment with mitomycin C (100 μg/ml in RPMI 1640/1% FBS for 1 hour at 37° C.) to inhibit APC proliferation, and 3 washes to remove residual mitomycin C.

Assays for induction of a T cell response are carried out in 96 well round bottom microtiter plates. Two to 4×10⁵ T cells are mixed with 2-4×10⁵ APC. Each T cell/APC combination is incubated, in triplicate, with and without OVA peptide (range 10-200 ng/well) for 3-5 days. Approximately 18 hr before termination of the culture 0.4 μCi of ³H-thymidine is added to each well. The wells are harvested using a Skatron cell harvester and ³H-thymidine incorporation (a measure of DNA synthesis and, therefore, T cell proliferation) is determined using a LKB liquid scintillation spectrometer.

A positive response is evident if the wells containing peptide incorporate significantly more ³H-Thymidine than those without peptide. Typically mice are considered positive where proliferation (in mean cpm) in response to peptide is more than 3 standard deviationis greater than the background proliferation without peptide. For each group, mean peptide specific proliferation is calculated by averaging values for each of the 3 mice.

Suppression of immunization will typically be considered as having occurred when the experimental group mean is greater than about 3 standard deviations less than the positive control group mean.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. A compound comprising:
(a) one or more MHC class I α3 complexes; and
(b) an antibody or a fragment thereof specific for a cell surface marker;
wherein said MHC class I α3 complexes comprise an isolated MHC class I α3 domain or fragment thereof, a β₂-microglobulin molecule or fragment thereof, and an antigenic peptide; and
wherein said MHC class I α3 complexes are linked to said antibody or fragment thereof.

2. The compound of claim 1, wherein said antigenic peptide is linked to said β₂-microglobulin molecule or fragment thereof.

3. The compound of claim 2, wherein said antigenic peptide is covalently bound to said β₂-microglobulin molecule or fragment thereof.

4. The compound of claim 1, wherein said β₂-microglobulin molecule or fragment thereof has been modified to have enhanced affinity for the intact MHC class I α chain relative to the isolated MHC class I α3 domain or fragment thereof.

5. The compound of claim 4, wherein said β₂-microglobulin molecule or fragment thereof has a serine to valine mutation at amino acid 55 of the mature protein.

6. The compound of claim 1, wherein said cell surface marker is a cell surface marker of a professional antigen presenting cell.

7. The compound of claim 6, wherein said professional antigen presenting cell is a dendritic cell.

8. The compound of claim 7, wherein said cell surface marker is selected from the group consisting of CD83, CMRF-44, CMRF-56, BCDA-2, BCDA-3, BCDA-4, and DEC-205.

9. The compound of claim 1, wherein said cell surface marker is a cell surface marker of a tumor cell.

10. The compound of claim 1, wherein said cell surface marker is a cell surface marker of an epithelial cell.

11. The compound of claim 1, wherein said cell surface marker is a cell surface marker of a fibroblast.

12. The compound of claim 1, wherein said cell surface marker is a cell surface marker of a T cell.

13. The compound of claim 12, wherein said cell surface marker is selected from the group consisting of CD28, CTLA-4 and CD25.

14. The compound of claim 1, wherein said cell surface marker is a cell surface marker of an infected cell.

15. The compound of claim 1, wherein said antigenic peptide is derived from a cancer cell.

16. The compound of claim 1, wherein said antigenic peptide is derived fromn an infectious agent or from infected cells.

17. The compound of claim 1, wherein said antigenic peptide is derived from the target tissue of an autoimmune disease.

18. The compound of claim 9, wherein said antigenic peptide is derived from a cancer cell.

19. The compound of claim 1, wherein said isolated MHC class I α3 domain or fragment thereof is linked to a carboxyl terminus of said antibody or fragment thereof.

20. A compound comprising:
(a) one or more MHC class I α3 complexes; and
(b) an antibody or a fragment thereof specific for a cell surface marker;
wherein said MHC class I α3 complexes comprise one ore more isolated MHC class I α3 domains or fragments thereof, a β₂-microglobulin molecule or fragment thereof, and a costimulatory molecule; and
wherein said MHC class I α3 complexes are linked to said antibody or fragment thereof.

21. The compound of claim 20, wherein said costimulatory molecule is linked to said β₂-microglobulin molecule or fragment thereof.

22. The compound of claim 21, wherein said costimulatory molecule is covalently bound to said β₂-microglobulin molecule or fragment thereof.

23. The compound of claim 20, wherein said β2-microglobulin molecule or fragment thereof has been modified to have enhanced affinity for the intact MHC class I α chain relative to the isolated MHC class I α3 domain thereof.

24. The compound of claim 20, wherein said β₂-microglobulin molecule or fragment thereof has a serine to valine mutation at amino acid 55 of the mature protein.

25. The compound of claim 20, wherein said cell surface marker is a cell surface marker of a professional antigen presenting cell.

26. The compound of claim 25, wherein said professional antigen presenting cell is a dendritic cell.

27. The compound of claim 26, wherein said cell surface marker is selected from the group consisting of CD83, CMRF-44, CMRF-56, BCDA-2, BCDA-3, BCDA-4, and DEC-205.

28. The compound of claim 20, wherein said cell surface marker is a cell surface marker of a tumor cell.

29. The compound of claim 20, wherein said cell surface marker is a cell surface marker of an epithelial cell.

30. The compound of claim 20, wherein said cell surface marker is a cell surface marker of a fibroblast.

31. The compound of claim 20, wherein said cell surface marker is a cell surface marker of a T cell.

32. The compound of claim 31, wherein said cell surface marker is selected from the group consisting of CD28, CTLA-4 and CD25.

33. The compound of claim 20, wherein said cell surface marker is a cell surface marker of an infected cell.

34. The compound of claim 20, wherein said costimulatory molecule is selected from the group consisting of B7.1 and B7.2.

35. The compound of claim 20, wherein said isolated MHC class I α3 domain or fragment thereof is linked to the carboxyl terminus of said antibody or fragment thereof.

36. A compound comprising:
(a) two or more MHC class I α3 complexes;
(b) a multivalent compound; and
(c) an antibody or a fragment thereof specific for a cell surface marker;
wherein said MHC class I α3 complexes comprise one or more isolated MHC class I α3 domains or fragment thereof, one or more β₂-microglobulins or fragment thereof, and one or more molecules selected from the group consisting of antigenic peptides, costimulatory molecules, and cytokines;
wherein said MHC class I α3 complexes are linked to said multivalent compound; and wherein said multivalent compound is linked to said antibody.

37. The compound of claim 36, wherein said one or more molecules are linked to said β₂-microglobulin or fragment thereof.

38. The compound of claim 37, wherein said one or more molecules are covalently bound to said β₂-microglobulin or fragment thereof.

39. The compound of claim 36, wherein said β₂-microglobulin molecule or fragment thereof has been modified to have enhanced affinity for the intact MHC class I α chain relative to the isolated MHC class I α3 domain thereof.

40. The compound of claim 36, wherein said β₂-microglobulin or fragment thereof has a serine to valine mutation at amino acid 55 of the mature protein.

41. The compound of claim 36, wherein said cell surface marker is a cell surface marker of a professional antigen presenting cell.

42. The compound of claim 41, wherein said professional antigen presenting cell is a dendritic cell.

43. The compound of claim 42, wherein said cell surface marker is selected from the group consisting of CD83, CMRF-44, CMRF-56, BCDA-2, BCDA-3, BCDA-4, and DEC-205.

44. The compound of claim 36, wherein said cell surface marker is a cell surface marker of a tumor cell.

45. The compound of claim 36, wherein said cell surface marker is a cell surface marker of an epithelial cell.

46. The compound of claim 36, wherein said cell surface marker is a cell surface marker of a fibroblast.

47. The compound of claim 36, wherein said cell surface marker is a cell surface marker of a T cell.

48. The compound of claim 47, wherein said cell surface marker is selected from the group consisting of CD28, CTLA-4 and CD25.

49. The compound of claim 36, wherein said antigenic peptide is derived from a cancer cell.

50. The compound of claim 36, wherein said antigenic peptide is derived from an infectious agent or from infected cells.

51. The compound of claim 36, wherein said antigenic peptide is derived from the target tissue of an autoimmune disease.

52. The compound of claim 36, comprising one ore more cytokines selected from the group consisting of B7.1 and B7.2.

53. The compound of claim 36, comprising one or more cytokines selected from the group consisting of: IL-1, IL-2, IL-3, IL-4, IL-5, IL-6, IL-7, IL-8, IL-9, IL-10, IL-11, IL-12, IL- 13, IL-14, IL-15, IL-16, IL-17, IL-18, α interferons, ω interferon, β interferons, γ interferons, τinterferon, colony stimulating, granulocyte- macrophage colony stimulating factor, transforming growth factor, and insulin-like growth factors.

54. The compound of claim 36, wherein said multivalent compound is avidin.

55. The compound of claim 36, wherein said multivalent compound is selected from the group consisting of streptavidin and chicken avidin.

56. The compound of claim 36, wherein said multivalent compound is a modified GCN4-zipper motif.

57. A polynucleotide encoding a compound comprising:
(a) one or more MHC class I α3 chains; and
(b) an antibody or fragment thereof specific for a cell surface marker;
wherein said MHC class I α3 chains are linked to said antibody or fragment thereof.

58. A method of immunizing an animal, comprising administering to said animal the compound of claim 1.

59. A method of immunizing an animal, comprising administering to said animal the compound of claim 20.

60. A method of immunizing an animal, comprising administering to said animal the compound of claim 36.


SEQUENCE LISTING (53 sequences)

SEQ ID NO: 1 - DNA, 276 bp, Homo sapiens
gacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgg

SEQ ID NO: 2 - DNA, 1075 bp, Artificial Sequence
  Assembled Ig Gamma Heavy Chain
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - DNA, 2003 bp, Artificial Sequence
  Ig alpha3 assembled chimera
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcggttcttcagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggaggcggttcttcagaattcggaggcggttcttcagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggaggcggttcttcaagatctggaggcggttcttcagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggtgacgggatcccg

SEQ ID NO: 4 - protein, 659 aa, Artificial Sequence
  Ig alpha3 assembled chimera
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSSDAPKTHMTHHAVSDHEATLRCWALSFYPAEITLTWQRDGEDQTQDTELVETRPAGDGTFQKWAAVVVPSGQEQRYTCHVQHEGLPKPLTLRWGGGSSEFGGGSSDAPKTHMTHHAVSDHEATLRCWALSFYPAEITLTWQRDGEDQTQDTELVETRPAGDGTFQKWAAVVVPSGQEQRYTCHVQHEGLPKPLTLRWGGGSSRSGGGSSDAPKTHMTHHAVSDHEATLRCWALSFYPAEITLTWQRDGEDQTQDTELVETRPAGDGTFQKWAAVVVPSGQEQRYTCHVQHEGLPKPLTLRW

SEQ ID NO: 5 - DNA, 448 bp, Artificial Sequence
  CMV peptide-beta2-microglobulin chimera
ccatcgatatgtctcgctccgtggccttagctgtgctcgcgctactctctctttctggcctggaggctaacctggtgcccatggtggctacggttggaggtgggggaggcggatcaggaggctcaggtgggtcaggaggcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 6 - protein, 143 aa, Artificial Sequence
  CMV peptide-beta2-microglobulin chimera
MSRSVALAVLALLSLSGLEANLVPMVATVGGGGGGSGGSGGSGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 7 - protein, 9 aa, Homo sapiens
SVAPPPEEV

SEQ ID NO: 8 - protein, 8 aa, Homo sapiens
VAPPPEEV

SEQ ID NO: 9 - protein, 8 aa, Homo sapiens
EVEPGSGV

SEQ ID NO: 10 - protein, 10 aa, Homo sapiens
EVEPGSGVRI

SEQ ID NO: 11 - protein, 8 aa, Homo sapiens
EATYLELA

SEQ ID NO: 12 - protein, 9 aa, Homo sapiens
ATYLELASA

SEQ ID NO: 13 - protein, 10 aa, Homo sapiens
ATYLELASAV

SEQ ID NO: 14 - protein, 8 aa, Homo sapiens
YLELASAV

SEQ ID NO: 15 - protein, 10 aa, Homo sapiens
SAVKEQYPGI

SEQ ID NO: 16 - protein, 9 aa, Homo sapiens
AVKEQYPGI

SEQ ID NO: 17 - protein, 8 aa, Homo sapiens
GIEIESRL

SEQ ID NO: 18 - protein, 9 aa, Homo sapiens
EIESRLGGT

SEQ ID NO: 19 - protein, 10 aa, Homo sapiens
RLGGTGAFEI

SEQ ID NO: 20 - protein, 9 aa, Homo sapiens
GTGAFEIEI

SEQ ID NO: 21 - protein, 8 aa, Homo sapiens
EIEINGQL

SEQ ID NO: 22 - protein, 9 aa, Homo sapiens
EIEINGQLV

SEQ ID NO: 23 - protein, 9 aa, Homo sapiens
DLIEAIRRA

SEQ ID NO: 24 - protein, 8 aa, Homo sapiens
LIEAIRRA

SEQ ID NO: 25 - protein, 10 aa, Homo sapiens
AIRRASNGET

SEQ ID NO: 26 - protein, 8 aa, Homo sapiens
RASNGETL

SEQ ID NO: 27 - protein, 10 aa, Homo sapiens
KITNSRPPCV

SEQ ID NO: 28 - protein, 9 aa, Homo sapiens
ITNSRPPCV

SEQ ID NO: 29 - protein, 10 aa, Homo sapiens
ITNSRPPCVI

SEQ ID NO: 30 - protein, 9 aa, Homo sapiens
EVEPGSGVR

SEQ ID NO: 31 - protein, 9 aa, Homo sapiens
EPCGFEATY

SEQ ID NO: 32 - protein, 9 aa, Homo sapiens
ASNGETLEK

SEQ ID NO: 33 - protein, 15 aa, Unknown
  Synthetic peptide linker
GGGGSGGGGSGGGGS

SEQ ID NO: 34 - protein, 15 aa, Unknown
  Synthetic peptide linker
GGGGSGGGGSGGGAS

SEQ ID NO: 35 - DNA, 10 bp, Unknown
  Synthetic oligonucleotide PCR primers
aatgctagcn

SEQ ID NO: 36 - DNA, 16 bp, Unknown
  Synthetic oligonucleotide PCR primers
atttctagaacttacn

SEQ ID NO: 37 - DNA, 28 bp, Unknown
  Synthetic oligonucleotide PCR primers
aattctagagtctgtccctaacatgccc

SEQ ID NO: 38 - DNA, 28 bp, Unknown
  Synthetic oligonucleotide PCR primers
aaaggtacctggaactgaggagcaggtg

SEQ ID NO: 39 - DNA, 30 bp, Unknown
  Synthetic oligonucleotide PCR primers
ggaggcggttcttcagacgcccccaaaacg

SEQ ID NO: 40 - DNA, 29 bp, Unknown
  Synthetic oligonucleotide PCR primers
cgggatcccgtcaccatctcagggtgagg

SEQ ID NO: 41 - DNA, 38 bp, Unknown
  Synthetic oligonucleotide PCR primers
ggaattctgaagaaccgcctccccatctcagggtgagg

SEQ ID NO: 42 - DNA, 37 bp, Unknown
  Synthetic oligonucleotide PCR primers
ggaattcggaggcggttcttcagacgcccccaaaacg

SEQ ID NO: 43 - DNA, 39 bp, Unknown
  Synthetic oligonucleotide PCR primers
tcagatcttgaagaaccgcctccccatctcagggtgagg

SEQ ID NO: 44 - DNA, 38 bp, Unknown
  Synthetic oligonucleotide PCR primers
gaagatctggaggcggttcttcagacgcccccaaaacg

SEQ ID NO: 45 - DNA, 38 bp, Unknown
  Synthetic oligonucleotide PCR primers
aattgcggccgcaaaccatgggatggagctgtatcatc

SEQ ID NO: 46 - DNA, 33 bp, Unknown
  Synthetic oligonucleotide PCR primers
tgaagaaccgcctcctttacccggagacaggga

SEQ ID NO: 47 - DNA, 83 bp, Unknown
  Synthetic oligonucleotide
ccatcgatatgtctcgctccgtggccttagctgtgctcgcgctactctctctttctggcctggaggctaacctggtgcccatg

SEQ ID NO: 48 - DNA, 83 bp, Unknown
  Synthetic oligonucleotide
catgggcaccaggttagcctccaggccagaaagagagagtagcgcgagcacagctaaggccacggagcgagacatatcgatgg

SEQ ID NO: 49 - DNA, 87 bp, Unknown
  Synthetic oligonucleotide
aacctggtgcccatggtggctacggttggaggtgggggaggcggatcaggaggctcaggtgggtcaggaggcatccagcgtactcca

SEQ ID NO: 50 - DNA, 87 bp, Unknown
  Synthetic oligonucleotide
tggagtacgctggatgcctcctgacccacctgagcctcctgatccgcctcccccacctccaaccgtagccaccatgggcaccaggtt

SEQ ID NO: 51 - DNA, 21 bp, Unknown
  Synthetic oligonucleotide
atccagcgtactccaaagatt

SEQ ID NO: 52 - DNA, 29 bp, Unknown
  Synthetic oligonucleotide
cgggatccttacatgtctcgatcccactt

SEQ ID NO: 53 - protein, 10 aa, Human immunodeficiency virus
RGPGRAFVTI